(19) United States
(12) Patent Application Publication
(10) Pub. No.: US 2002/0062500 A1
(43) Pub. Date: May 23, 2002

(54) Hypersensitive response eliciting domains and use thereof

(76) Inventors: Hao Fan, Bothell, WA (US); Zhong-Min Wei, Kirkland, WA (US)

Correspondence Address: Michael L. Goldman, NIXON PEABODY LLP, Clinton Square, P.O. Box 31051, Rochester, NY, 14603, US

(21) Appl. No.: 09/879,248

(22) Filed: Jun. 12, 2001

Related U.S. Application Data
(60) Provisional application No. 60/212,211, filed on Jun. 16, 2000.

(51) Int. Cl. A01H 5/00; C07H 21/04; C12N 9/00; C07K 14/415

(52) U.S. Cl. 800/288; 800/279; 435/183; 435/410; 536/23.4; 530/350

(57) ABSTRACT

The present invention is directed to the structure of an isolated protein or polypeptide which elicits a hypersensitive response in plants as well as an isolated nucleic acid molecule which encodes the hypersensitive response eliciting protein or polypeptide. This protein or polypeptide has an acid portion linked to an alpha helix or a pair of spaced apart domains comprising an acidic portion linked to an alpha-helix. This isolated protein or polypeptide and the isolated nucleic acid molecule can used to impart disease resistance to plants, to enhance plant growth, to control insects, and/or to impart stress resistance to plants. This can be achieved by applying the hypersensitive response elicitor protein or polypeptide in a non-infectious form to plants or plant seeds under conditions effective to impart disease resistance, to enhance plant growth, to control insects, and/or to impart stress resistance to plants or plants grown from the plant seeds. Alternatively, transgenic plants or plant seeds transformed with a nucleic acid molecule encoding a hypersensitive response elicitor protein or polypeptide can be provided and the transgenic plants or plants resulting from the transgenic plant seeds are grown under conditions effective to impart disease resistance, to enhance plant growth, to control insects, and/or to impart stress resistance to plants or plants grown from the plant seeds.

This application claims benefit of U.S. Provisional Patent Application Ser. No. 60/212,211, filed on Jun. 16, 2000.

### FIELD OF THE INVENTION

The present invention relates to hypersensitive response elicitors and their structure.

### BACKGROUND OF THE INVENTION

Interactions between bacterial pathogens and their plant hosts generally fall into two categories: (1) compatible (pathogen-host), leading to intercellular bacterial growth, symptom development, and disease development in the host plant; and (2) incompatible (pathogen-nonhost), resulting in the hypersensitive response, a particular type of incompatible interaction occurring, without progressive disease symptoms. During compatible interactions on host plants, bacterial populations increase dramatically and progressive symptoms occur. During incompatible interactions, bacterial populations do not increase, and progressive symptoms do not occur.

The hypersensitive response is a rapid, localized necrosis that is associated with the active defense of plants against many pathogens (Kiraly, Z., “Defenses Triggered by the Invader: Hypersensitivity,” pages 201-224 in: Plant Disease: An Advanced Treatise, Vol. 5, J. G. Horsfall and E. B. Cowling, ed. Academic Press New York (1980); Klement, Z., “Hypersensitivity,” pages 149-177 in: Phytopathogenic Prokaryotes, Vol. 2, M.S. Mount and G. H. Lacy, ed. Academic Press, New York (1982)). The hypersensitive response elicited by bacteria is readily observed as a tissue collapse if high concentrations (≧10⁷ cells/ml) of a limited host-range pathogen like Pseudomonas syringae or Erwinia amylovora are infiltrated into the leaves of nonhost plants (necrosis occurs only in isolated plant cells at lower levels of inoculum) (Klement, Z., “Rapid Detection of Pathogenicity of Phytopathogenic Pseudomonads,” Nature 199:299-300; Klement, et al., “Hypersensitive Reaction Induced by Phytopathogenic Bacteria in the Tobacco Leaf” Phytopathology 54:474-477 (1963); Turner, et al., “The Quantitative Relation Between Plant and Bacterial Cells Involved in the Hypersensitive Reaction,” Phytopathology 64:885-890 (1974); Klement, Z., “Hypersensitivity,” pages 149-177 in Phytopathogenic Prokaryotes, Vol. 2., M.S. Mount and G. H. Lacy, ed. Academic Press, New York (1982)). The capacities to elicit the hypersensitive response in a nonhost and be pathogenic in a host appear linked. As noted by Klement, Z., “Hypersensitivity,” pages 149-177 in Phytopathogenic Prokaryotes, Vol. 2., M. S. Mount and G. H. Lacy, ed. Academic Press, New York, these pathogens also cause physiologically similar, albeit delayed, necroses in their interactions with compatible hosts. Furthermore, the ability to produce the hypersensitive response or pathogenesis is dependent on a common set of genes, denoted hrp (Lindgren, P. B., et al., “Gene Cluster of Pseudomonas syringae pv. ‘phaseolicola’ Controls Pathogenicity of Bean Plants and Hypersensitivity on Nonhost Plants,” J. Bacteriol. 168:512-22 (1986); Willis, D. K., et al., “hrp Genes of Phytopathogenic Bacteria,” Mol. Plant-Microbe Interact. 4:132-138 (1991)). Consequently, the hypersensitive response may hold clues to both the nature of plant defense and the basis for bacterial pathogenicity.

The hrp genes are widespread in gram-negative plant pathogens, where they are clustered, conserved, and in some cases interchangeable (Willis, D. K., et al., “hrp Genes of Phytopathogenic Bacteria,” Mol. Plant-Microbe Interact. 4:132-138 (1991); Bonas, U., “hrp Genes of Phytopathogenic Bacteria,” pages 79-98 in: Current Topics in Microbiology and Immunology: Bacterial Pathogenesis of Plants and Animals—Molecular and Cellular Mechanisms, J. L. Dangl, ed. Springer-Verlag, Berlin (1994)). Several hrp genes encode components of a protein secretion pathway similar to one used by Yersinia, Shigella, and Salmonella spp. to secrete proteins essential in animal diseases (Van Gijsegem, et al., “Evolutionary Conservation of Pathogenicity Determinants Among Plant and Animal Pathogenic Bacteria,” Trends Microbiol. 1:175-180 (1993)). In E. amylovora, P. syringae, and P. solanacearum, hrp genes have been shown to control the production and secretion of glycine-rich, protein elicitors of the hypersensitive response (He, S. Y., et al. “Pseudomonas Syringae pv. Syringae HarpinPss: a Protein that is Secreted via the Hrp Pathway and Elicits the Hypersensitive Response in Plants,” Cell 73:1255-1266 (1993), Wei, Z.-H., et al., “HrpI of Erwinia amylovora Functions in Secretion of Harpin and is a Member of a New Protein Family,” J. Bacteriol. 175:7958-7967 (1993); Arlat, M. et al. “PopA1, a Protein Which Induces a Hypersensitive-like Response on Specific Petunia Genotypes, is Secreted via the Hrp Pathway of Pseudomonas solanacearum,” EMBO J. 13:543-553 (1994)).

The first of these proteins was discovered in E. amylovora Ea321, a bacterium that causes fire blight of rosaceous plants, and was designated harpin (Wei, Z.-M., et al, “Harpin, Elicitor of the Hypersensitive Response Produced by the Plant Pathogen Erwinia amylovora,” Science 257:85-88 (1992)). Mutations in the encoding hrpN gene revealed that harpin is required for E. amylovora to elicit a hypersensitive response in nonhost tobacco leaves and incite disease symptoms in highly susceptible pear fruit. The P. solanacearum GMI1000 PopA1 protein has similar physical properties and also elicits the hypersensitive response in leaves of tobacco, which is not a host of that strain (Arlat, et al. “PopA1, a Protein Which Induces a Hypersensitive-like Response on Specific Petunia Genotypes, is Secreted via the Hrp Pathway of Pseudomonas solanacearum,” EMBO J. 13:543-53 (1994)). However, P. solanacearum popA mutants still elicit the hypersensitive response in tobacco and incite disease in tomato. Thus, the role of these glycine-rich hypersensitive response elicitors can vary widely among gram-negative plant pathogens.

Other plant pathogenic hypersensitive response elicitors have been isolated, cloned, and sequenced. These include: Erwinia chrysanthemi (Bauer, et. al., “Erwinia chrysanthemi Harpin_{Ech}: Soft-Rot Pathogenesis,” MPMI 8(4): 484-91 (1995)); Erwinia carotovora (Cui, et. al., “The RsmA^{—}Mutants of Erwinia carotovora subsp. carotovora Strain Ecc71 Overexpress hrpN_{Ecc} and Elicit a Hypersensitive Reaction-like Response in Tobacco Leaves,” MPMI 9(7): 565-73 (1966)); Erwinia stewartii (Ahlmad, et. al., “Harpin is not Necessary for the Pathogenicity of Erwinia stewartii on Maize,” 8th Int'l. Cong. Molec. Plant-Microb. Inter. July 14-19, 1996 and Ahmad, et. al., “Harpin is not Necessary for the Pathogenicity of Erwinia stewartii on Maize,” Ann. Mtg. Am. Phytopath. Soc. July 27-31, 1996); and Pseudomonas syringae pv. syringae (WO 94/26782 to Cornell Research Foundation, Inc.).

The present invention is a further advance in the effort to identify and characterize hypersensitive response elicitor proteins.

### SUMMARY OF THE INVENTION

One aspect of the present invention is directed to an isolated hypersensitive response elicitor protein comprising a pair of spaced apart domains, with each comprising an acid portion linked to an alpha-helix.

Another embodiment of the present invention relates to an isolated hypersensitive response elicitor protein comprising an acid portion linked to an alpha-helix.

Nucleic acid molecules encoding either of these proteins as well as vectors, host cells, transgenic plants, and transgenic plant seeds containing those nucleic acid molecules are also disclosed.

The protein of the present invention can be used to impart disease resistance to plants, to enhance plant growth, to control insects, and/or impart stress resistance. This involves applying the protein to plants or plant seeds under conditions effective to impart disease resistance, to enhance plant growth, to control insects, and/or impart stress resistance to plants or plants grown from the plant seeds.

As an alternative to applying the protein to plants or plant seeds in order to impart disease resistance, to enhance plant growth, to control insects on plants, and/or impart stress resistance, transgenic plants or plant seeds can be utilized. When utilizing transgenic plants, this involves providing a transgenic plant transformed with a nucleic acid molecule encoding the protein of the present invention and growing the plant under conditions effective to impart disease resistance, to enhance plant growth, to control insects, and/or to impart stress resistance to the plants or plants grown from the plant seeds. Alternatively, a transgenic plant seed transformed with the nucleic acid molecule encoding the protein of the present invention can be provided and planted in soil. A plant is then propagated under conditions effective to impart disease resistance, to enhance plant growth, to control insects, and/or to impart stress resistance to plants or plants grown from the plant seeds.

### BRIEF DESCRIPTION OF THE DRAWINGS

FIG. 1 is a schematic drawing showing the construction of a universal expression cassette for a hypersensitive response domain.

### DETAILED DESCRIPTION OF THE INVENTION

The present invention is directed to an isolated hypersensitive response elicitor protein comprising a pair of spaced apart domains, with each comprising an acid portion linked to an alpha-helix. The acidic portion is a polypeptide with 10 or more amino acids, is rich in acidic amino acids, and has a pI below 5.0. The acidic portion has a secondary structure in the form of a beta-sheet or a beta-turn. The secondary structure of this unit can also be in an unordered form.

The alpha-helix portion of the present invention is a polypeptide with 10 or more amino acids. Its secondary structure is in the form of a stable alpha-helix.

Another embodiment of the present invention relates to an isolated hypersensitive response elicitor protein comprising an acid portion linked to an alpha-helix.

Both of these proteins are capable of eliciting a hypersensitive response.

The alpha helix is a common structural motif of proteins in which a linear sequence of amino acid folds into a right-handed helix stabilized by internal hydrogen bonding between backbone atoms.

The acidic motif includes a certain combination of amino acids in which a linear sequence with a pI below 5.0 folds into a β sheet, coil, or thin structures but not an alpha helix of secondary structure.

The hypersensitive response elicitor polypeptides or proteins according to the present invention can be derived from hypersensitive response elicitor polypeptides or proteins of a wide variety of fungal and bacterial pathogens. Such polypeptides or proteins are able to elicit local necrosis in plant tissue contacted by the elicitor. Examples of suitable bacterial sources of polypeptide or protein elicitors include Erwinia, Pseudomonas, and Xanthamonas species (e.g., the following bacteria: Erwinia amylovora, Erwinia chrysanthemi, Erwinia stewartii, Erwinia carotovora, Pseudomonas syringae, Pseudomonas solancearum, Xanthomonas campestris, and mixtures thereof). In addition to hypersensitive response elicitors from these Gram negative bacteria, it is possible to use elicitors from Gram positive bacteria. One example is Clavibacter michiganensis subsp. sepedonicus.

An example of a fungal source of a hypersensitive response elicitor protein or polypeptide is Phytophthora. Suitable species of Phytophthora include Phytophthora parasitica, Phytophthora cryptogea, Phytophthora cinnamomi, Phytophthora capsici, Phytophthora megasperma, and Phytophthora citrophthora.

The hypersensitive response elicitor polypeptide or protein from Erwinia chrysanthemi has an amino acid sequence corresponding to SEQ. ID. No. 1 as follows:

```
Met Gln Ile Thr Ile Lys Ala His Ile Gly Gly Asp Leu Gly Val Ser
1 5 10 15
Gly Leu Gly Ala Gln Gly Leu Lys Gly Leu Asn Ser Ala Ala Ser Ser
                        20 25 30
Leu Gly Ser Ser Val Asp Lys Leu Ser Ser Thr Ile Asp Lys Leu Thr
                35 40 45
Ser Ala Leu Thr Ser Met Met Phe Gly Gly Ala Leu Ala Gln Gly Leu
        50 55 60
Gly Ala Ser Ser Lys Gly Leu Gly Met Ser Asn Gln Leu Gly Gln Ser
65 70 75 80
Phe Gly Asn Gly Ala Gln Gly Ala Ser Asn Leu Leu Ser Val Pro Lys
                                85 90 95
Ser Gly Gly Asp Ala Leu Ser Lys Met Phe Asp Lys Ala Leu Asp Asp
                        100 105 110
Leu Leu Gly His Asp Thr Val Thr Lys Leu Thr Asn Gln Ser Asn Gln
                115 120 125
Leu Ala Asn Ser Met Leu Asn Ala Ser Gln Met Thr Gln Gly Asn Met
        130 135 140
Asn Ala Phe Gly Ser Gly Val Asn Asn Ala Leu Ser Ser Ile Leu Gly
145 150 155 160
Asn Gly Leu Gly Gln Ser Met Ser Gly Phe Ser Gln Pro Ser Leu Gly
                                165 170 175
Ala Gly Gly Leu Gln Gly Leu Ser Gly Ala Gly Ala Phe Asn Gln Leu
                        180 185 190
Gly Asn Ala Ile Gly Met Gly Val Gly Gln Asn Ala Ala Leu Ser Ala
                195 200 205
Leu Ser Asn Val Ser Thr His Val Asp Gly Asn Asn Arg His Phe Val
        210 215 220
Asp Lys Glu Asp Arg Gly Met Ala Lys Glu Ile Gly Gln Phe Met Asp
225 230 235 240
Gln Tyr Pro Glu Ile Phe Gly Lys Pro Glu Tyr Gln Lys Asp Gly Trp
                                245 250 255
Ser Ser Pro Lys Thr Asp Asp Lys Ser Trp Ala Lys Ala Leu Ser Lys
                        260 265 270
Pro Asp Asp Asp Gly Met Thr Gly Ala Ser Met Asp Lys Phe Arg Gln
                275 280 285
Ala Met Gly Met Ile Lys Ser Ala Val Ala Gly Asp Thr Gly Asn Thr
        290 295 300
Asn Leu Asn Leu Arg Gly Ala Gly Gly Ala Ser Leu Gly Ile Asp Ala
305 310 315 320
Ala Val Val Gly Asp Lys Ile Ala Asn Met Ser Leu Gly Lys Leu Ala
                                325 330 335
Asn Ala
```

This hypersensitive response elicitor polypeptide or protein has a molecular weight of 34 kDa, is heat stable, has a glycine content of greater than 16%, and contains substantially no cysteine. The Erwinia chrysanthemi hypersensitive response elicitor polypeptide or protein is encoded by a DNA molecule having a nucleotide sequence corresponding to SEQ. ID. No. 2 as follows:

| | |
|---|---|
| CGATTTTACC CGGGTGAACG TGCTATGACC GACAGCATCA CGGTATTCGA CACCGTTACG | 60 |
| GCGTTTATGG CCGCGATGAA CCGGCATCAG GCGGCGCGCT GGTCGCCGCA ATCCGGCGTC | 120 |
| GATCTGGTAT TTCAGTTTGG GGACACCGGG CGTGAACTCA TGATGCAGAT TCAGCCGGGG | 180 |
| CAGCAATATC CCGGCATGTT GCGCACGCTG CTCGCTCGTC GTTATCAGCA GGCGGCAGAG | 240 |
| TGCGATGGCT GCCATCTGTG CCTGAACGGC AGCGATGTAT TGATCCTCTG GTGGCCGCTG | 200 |
| CCGTCGGATC CCGGCAGTTA TCCGCAGGTG ATCGAACGTT TGTTTGAACT GGCGGGAATG | 360 |
| ACGTTGCCGT CGCTATCCAT AGCACCGACG GCGCGTCCGC AGACAGGGAA CGGACGCGCC | 420 |
| CGATCATTAA GATAAAGGCG GCTTTTTTTA TTGCAAAACG GTAACGGTGA GGAACCGTTT | 480 |
| CACCGTCGGC GTCACTCAGT AACAAGTATC CATCATGATG CCTACATCGG GATCGGCGTG | 540 |
| GGCATCCGTT GCAGATACTT TTGCGAACAC CTGACATGAA TGAGGAAACG AAATTATGCA | 600 |
| AATTACGATC AAAGCGCACA TCGGCGGTGA TTTGGGCGTC TCCGGTCTGG GGCTGGGTGC | 660 |
| TCAGGGACTG AAAGGACTGA ATTCCGCGGC TTCATCGCTG GGTTCCAGCG TGGATAAACT | 720 |
| GAGCAGCACC ATCGATAAGT TGACCTCCGC GCTGACTTCG ATGATGTTTG GCGGCGCGCT | 780 |
| GGCGCAGGGG CTGGGCGCCA GCTCGAAGGG GCTGGGGATG AGCAATCAAC TCGGCCAGTC | 840 |
| TTTCGGCAAT GGCGCGCAGG GTGCGAGCAA CCTGCTATCC GTACCGAAAT CCGGCGGCGA | 900 |
| TGCGTTGTCA AAAATGTTTG ATAAAGCGCT GGACGATCTG CTGGGTCATG ACACCGTGAC | 960 |
| CAAGCTGACT AACCAGAGCA ACCAACTGGC TAATTCAATG CTGAACGCCA GCCAGATGAC | 1020 |
| CCAGGGTAAT ATGAATGCGT TCGGCAGCGG TGTGAACAAC GCACTGTCGT CCATTCTCGG | 1080 |
| CAACGGTCTC GGCCAGTCGA TGAGTGGCTT CTCTCAGCCT TCTCTGGGGG CAGGCGGCTT | 1140 |
| GCAGGGCCTG AGCGGCGCGG GTGCATTCAA CCAGTTGGGT AATGCCATCG GCATGGGCGT | 1200 |
| GGGGCAGAAT GCTGCGCTGA GTGCGTTGAG TAACGTCAGC ACCCACGTAG ACGGTAACAA | 1260 |
| CCGCCACTTT GTAGATAAAG AAGATCGCGG CATGGCGAAA GAGATCGGCC AGTTTATGGA | 1320 |
| TCAGTATCCG GAAATATTCG GTAAACCGGA ATACCAGAAA GATGGCTGGA GTTCGCCGAA | 1380 |
| GACGGACGAC AAATCCTGGG CTAAAGCGCT GAGTAAACCG GATGATGACG GTATGACCGG | 1440 |
| CGCCAGCATG GACAAATTCC GTCAGGCGAT GGGTATGATC AAAAGCGCGG TGGCGGGTGA | 1500 |
| TACCGGCAAT ACCAACCTGA ACCTGCGTGG CGCGGGCCGT CCATCGCTGG GTATCGATGC | 1560 |
| GGCTGTCGTC GGCGATAAAA TAGCCAACAT GTCGCTGGGT AACCTGGCCA ACGCCTGATA | 1620 |
| ATCTGTGCTG GCCTGATAAA GCGGAAACGA AAAAAGAGAC GCGCAAGCCT GTCTCTTTTC | 1680 |
| TTATTATGCG GTTTATGCGG TTACCTGGAC CGGTTAATCA TCGTCATCGA TCTGGTACAA | 1740 |
| ACGCACATTT TCCCGTTCAT TCGCGTCGTT ACGCGCCACA ATCGCGATGG CATCTTCCTC | 1800 |
| GTCGCTCAGA TTGCGCGGCT GATGGGGAAC GCCGGGTGGA ATATAGAGAA ACTCGCCGCC | 1860 |
| CAGATGGAGA CACGTCTGCG ATAAATCTGT GCCGTAACGT GTTTCTATCC GCCCCTTTAG | 1920 |
| CAGATAGATT GCGGTTTCGT AATCAACATG GTAATGCGGT TCCGCCTGTG CGCCGGCCGG | 1980 |
| GATCACCACA ATATTCATAG AAAGCTGTCT TGCACCTACC GTATCGCGGG AGATACOGAC | 2040 |
| AAAATAGGGC AGTTTTTGCG TGGTATCCGT GGGGTGTTCC GGCCTGACAA TCTTGAGTTG | 2100 |
| GTTCGTCATC ATCTTTCTCC ATCTGGGCGA CCTGATCGGT T | 2141 |

The hypersensitive response elicitor from Erwinia chrysanthemi has 2 hypersensitive response eliciting domains. The first domain extends, within SEQ. ID. No. 1, from amino acid 69 to amino acid 122, particularly from amino acid 85 to amino acid 116. The acidic unit in the first domain extends, within SEQ. ID. No. 1, from amino acid 69 to amino acid 102, particularly from amino acid 85 to amino acid 102. The alpha-helix in the first domain extends, within SEQ. ID. No. 1, from amino acid 102 to amino acid 122, particularly from amino acid 102 to amino acid 116. The second domain extends, within SEQ. ID. No. 1, from amino acid 251 to amino acid 299, particularly from amino acid 256 to amino acid 292. The acidic unit in the second domain extends, within SEQ. ID. No. 1, from amino acid 251 to amino acid 279, particularly from amino acid 261 to amino acid 279. The alpha-helix in the second domain extends, within SEQ. ID. No. 1, from amino acid 279 to amino acid 299, particularly from amino acid 279 to amino acid 292.

The hypersensitive response elicitor polypeptide or protein derived from Erwinia amylovora has an amino acid sequence corresponding to SEQ. ID. No. 3 as follows:

```
Met Ser Leu Asn Thr Ser Gly Leu Gly Ala Ser Thr Met Gln Ile Ser
1 5 10 15
Ile Gly Gly Ala Gly Gly Asn Asn Gly Leu Leu Gly Thr Ser Arg Gln
                        20 25 30
Asn Ala Gly Leu Gly Gly Asn Ser Ala Leu Gly Leu Gly Gly Gly Asn
                35 40 45
Gln Asn Asp Thr Val Asn Gln Leu Ala Gly Leu Leu Thr Gly Met Met
        50 55 60
Met Met Met Ser Met Met Gly Gly Gly Gly Leu Met Gly Gly Gly Leu
65 70 75 80
Gly Gly Gly Leu Gly Asn Gly Leu Gly Gly Ser Gly Gly Leu Gly Glu
                                85 90 95
Gly Leu Ser Asn Ala Leu Asn Asp Met Leu Gly Gly Ser Leu Asn Thr
                        100 105 110
Leu Gly Ser Lys Gly Gly Asn Asn Thr Thr Ser Thr Thr Asn Ser Pro
                115 120 125
Leu Asp Gln Ala Leu Gly Ile Asn Ser Thr Ser Gln Asn Asp Asp Ser
        130 135 140
Thr Ser Gly Thr Asp Ser Thr Ser Asp Ser Ser Asp Pro Met Gln Gln
145 150 155 160
Leu Leu Lys Met Phe Ser Glu Ile Met Gln Ser Leu Phe Gly Asp Gly
                                165 170 175
Gln Asp Gly Thr Gln Gly Ser Ser Ser Gly Gly Lys Gln Pro Thr Glu
                        180 185 190
Gly Glu Gln Asn Ala Tyr Lys Lys Gly Val Thr Asp Ala Leu Ser Gly
                195 200 205
Leu Met Gly Asn Gly Leu Ser Gln Leu Leu Gly Asn Gly Gly Leu Gly
        210 215 220
Gly Gly Gln Gly Gly Asn Ala Gly Thr Gly Leu Asp Gly Ser Ser Leu
225 230 235 240
Gly Gly Lys Gly Leu Gln Asn Leu Ser Gly Pro Val Asp Tyr Gln Gln
                                245 250 255
Leu Gly Asn Ala Val Gly Thr Gly Ile Gly Met Lys Ala Gly Ile Gln
                        260 265 270
Ala Leu Asn Asp Ile Gly Thr His Arg His Ser Ser Thr Arg Ser Phe
                275 280 285
Val Asn Lys Gly Asp Arg Ala Met Ala Lys Glu Ile Gly Gln Phe Met
        290 295 300
Asp Gln Tyr Pro Glu Val Phe Gly Lys Pro Gln Tyr Gln Lys Gly Pro
305 310 315 320
Gly Gln Glu Val Lys Thr Asp Asp Lys Ser Trp Ala Lys Ala Leu Ser
                                325 330 335
Lys Pro Asp Asp Asp Gly Met Thr Pro Ala Ser Met Glu Gln Phe Asn
                        340 345 350
Lys Ala Lys Gly Met Ile Lys Arg Pro Met Ala Gly Asp Thr Gly Asn
                355 360 365
Gly Asn Leu Gln Ala Arg Gly Ala Gly Gly Ser Ser Leu Gly Ile Asp
        370 375 380
Ala Met Met Ala Gly Asp Ala Ile Asn Asn Met Ala Leu Gly Lys Leu
385 390 395 400
Gly Ala Ala
```

This hypersensitive response elicitor polypeptide or protein has a molecular weight of about 39 kDa, has a pI of approximately 4.3, and is heat stable at 100° C. for at least 10 minutes. This hypersensitive response elicitor polypeptide or protein has substantially no cysteine. The hypersensitive response elicitor polypeptide or protein derived from Erwinia amylovora is more fully described in Wei, Z.-M., R. J. Laby, C. H. Zumoff, D. W. Bauer, S.-Y. He, A. Collmer, and S. V. Beer, “Harpin, Elicitor of the Hypersensitive Response Produced by the Plant Pathogen Erwinia amylovora,” Science 257:85-88 (1992), which is hereby incorporated by reference. The DNA molecule encoding this polypeptide or protein has a nucleotide sequence corresponding to SEQ. ID. No. 4 as follows:

| | |
|---|---|
| AAGCTTCGGC ATGGCACGTT TGACCGTTGG GTCGGCAGGG TACGTTTGAA TTATTCATAA | 60 |
| GAGGAATACG TTATGAGTCT GAATACAAGT GGGCTGGGAG CGTCAACGAT GCAAATTTCT | 120 |
| ATCGGCGGTG CGGGCGGAAA TAACGGGTTG CTGGGTACCA GTCGCCAGAA TGCTGGGTTG | 180 |
| GGTGGCAATT CTGCACTGGG GCTGGGCGGC GGTAATCAAA ATGATACCGT CAATCAGCTG | 240 |
| GCTGGCTTAC TCACCGGCAT GATGATGATG ATGAGCATGA TGGGCGGTGG TGGGCTGATG | 300 |
| GGCGGTGGCT TAGGCGGTGG CTTAGGTAAT GGCTTCGGTG GCTCAGGTGG CCTGGGCGAA | 360 |
| GGACTGTCGA ACGCGCTGAA CGATATGTTA GGCGGTTCGC TGAACACGCT GGGCTCGAAA | 420 |
| GGCGGCAACA ATACCACTTC AACAACAAAT TCCCCGCTGG ACCAGGCGCT GGGTATTAAC | 480 |
| TCAACGTCCC AAAACGACGA TTCCACCTCC GGCACAGATT CCACCTCAGA CTCCAGCGAC | 540 |
| CCGATGCAGC AGCTGCTGAA GATGTTCAGC GAGATAATGC AAAGCCTGTT TGGTGATGGG | 600 |
| CAAGATGGCA CCCAGGGCAG TTCCTCTGGG GGCAAGCAGC CGACCGAAGG CGAGCAGAAC | 660 |
| GCCTATAAAA AAGGAGTCAC TGATGCGCTG TCGGGCCTGA TGGGTAATGG TCTGAGCCAG | 720 |
| CTCCTTGGCA ACGGGGGACT GGGAGGTGGT CAGGGCGGTA ATGCTGGCAC GGGTCTTGAC | 780 |
| GGTTCGTCGC TGGGCGGCAA AGGGCTGCAA AACCTGAGCG GGCCGGTGGA CTACCAGCAG | 840 |
| TTAGGTAACG CCGTGGGTAC CGGTATCGGT ATGAAAGCGG GCATTCAGGC GCTGAATGAT | 900 |
| ATCGGTACGC ACAGGCACAG TTCAACCCGT TCTTTCGTCA ATAAAGGCGA TCGGGCGATG | 960 |
| GCGAAGGAAA TCGGTCAGTT CATGGACCAG TATCCTGAGG TGTTTGGCAA GCCGCAGTAC | 1020 |
| CAGAAAGGCC CGGGTCAGGA GGTGAAAACC GATGACAAAT CATGGGCAAA AGCACTGAGC | 1080 |
| AAGCCAGATG ACGACGGAAT GACACCAGGC AGTATGGAGC AGTTCAACAA AGCCAAGGGC | 1140 |
| ATGATCAAAA GGCCCATGGC GGGTGATACC GGCAACGGCA ACCTGCAGGC ACGCGGTGCC | 1200 |
| GGTGGTTCTT CGCTGGGTAT TGATGCCATG ATGGCCGGTG ATGCCATTAA CAATATGGCA | 1288 |
| CTTGGCAAGC TGGGCGCGGC TTAAGCTT | 1288 |

The hypersensitive response elicitor from Erwinia amylovora has 2 hypersensitive response eliciting domains. The first domain extends, within SEQ. ID. No. 3, from amino acid 32 to amino acid 74, particularly from amino acid 45 to amino acid 68. The acidic unit in the first domain extends, within SEQ. ID. No. 3, from amino acid 32 to amino acid 57, particularly from amino acid 45 to amino acid 57. The alpha-helix in the first domain extends, within SEQ. ID. No. 3, from amino acid 57 to amino acid 74, particularly from amino acid 57 to amino acid 68. The second domain extends, within SEQ. ID. No. 3, from amino acid 130 to amino acid 180, particularly from amino acid 145 to amino acid 170. The acidic unit in the second domain extends, within SEQ. ID. No. 3, from amino acid 130 to amino acid 157, particularly from amino acid 145 to amino acid 157. The alpha-helix in the second domain extends, within SEQ. ID. No. 3, from amino acid 157 to amino acid 180, particularly from amino acid 157 to amino acid 170.

Another potentially suitable hypersensitive response elicitor from Erwinia amylovora is disclosed in U.S. patent application Ser. No. 09/120,927, which is hereby incorporated by reference. The protein is encoded by a DNA molecule having a nucleic acid sequence of SEQ. ID. No. 5 as follows:

| | |
|---|---|
| ATGTCAATTC TTACGCTTAA CPACAATACC TCGTCCTCGC CGGGTCTGTT CCAGTCCGGG | 60 |
| GGGGACAACG GGCTTGGTGG TCATAATGCA AATTCTGCGT TGGGGCAACA ACCCATCGAT | 120 |
| CGGCAAACCA TTGAGCAAAT GGCTCAATTA TTGGCGGAAC TGTTAAAGTC ACTGCTATCG | 180 |
| CCACAATCAC GTAATGCGGC AACCGGAGCC GGTGGCAATG ACCAGACTAC AGGAGTTGGT | 240 |
| AACGCTGGCG GCCTGAACGG ACGAAAAGGC ACAGCAGGAA CCACTCCGCA GTCTGACAGT | 300 |
| CAGAACATGC TGAGTGAGAT GGGCAACAAC GGGCTGGATC AGGCCATCAC GCCCGATGGC | 360 |
| CAGGGCGGCG GGCAGATCGG CGATAATCCT TTACTGAAAG CCATGCTGAA GCTTATTGCA | 420 |
| CGCATGATGG ACGGCCAAAG CGATCAGTTT GGCCAACCTG GTACGGGCAA CAACAGTGCC | 480 |
| TCTTCCGGTA CTTCTTCATC TGGCGGTTCC CCTTTTAACG ATCTATCAGG GGGGAAGGCC | 540 |
| CCTTCCGGCA ACTCCCCTTC CGGCAACTAC TCTCCCGTCA GTACCTTCTC ACCCCCATCC | 600 |
| ACGCCAACGT CCCCTACCTC ACCGCTTGAT TTCCCTTCTT CTCCCACCAA AGCAGCCGGG | 660 |
| GGCAGCACGC CGGTAACCGA TCATCCTCAC CCTGTTGGTA GCGCGGGCAT CGGGGCCGGA | 720 |
| AATTCGGTGG CCTTCACCAG CGCCGGCGCT AATCAGACGG TGCTGCATGA CACCATTACC | 780 |
| GTGAAAGCGG GTCAGGTGTT TGATGGCAAA GGACAAACCT TCACCGCCCG TTCAGAATTA | 840 |
| GGCGATGGCC GCCAGTCTGA AAACCACAAA CCGCTGTTTA TACTGGAAGA CGGTGCCAGC | 900 |
| CTGAAAAACG TCACCATGGG CGACGACGGG GCGGATGGTA TTCATCTTTA CGGTGATGCC | 960 |
| AAAATAGACA ATCTGCACGT CACCAACGTG GGTGAGGACG CGATTACCGT TAAGCCAAAC | 1020 |
| AGCGCGGGCA AAAAATCCCA CGTTGAAATC ACTAACAGTT CCTTCGAGCA CGCCTCTGAC | 1080 |
| AAGATCCTGC AGCTGAATGC CGATACTAAC CTGAGCGTTG ACAACGTGAA GGCCAAAGAC | 1140 |
| TTTGGTACTT TTGTACGCAC TAACGGCGGT CAACAGGGTA ACTGCGATCT GAATCTGAGC | 1200 |
| CATATCAGCG CAGAAGACGG TAAGTTCTCG TTCGTTAAAA GCGATAGCGA GGGGCTAAAC | 1260 |
| GTCAATACCA GTGATATCTC ACTGGGTGAT GTTGAAAACC ACTACAAAGT GCCGATGTCC | 1320 |
| GCCAACCTGA AGGTGGCTGA ATGA | 1344 |

See GenBank Accession No. U945 13. The isolated DNA molecule of the present invention encodes a hypersensitive response elicitor protein or polypeptide having an amino acid sequence of SEQ. ID. No. 6 as follows:

```
Met Ser Ile Leu Thr Leu Asn Asn Asn Thr Ser Ser Ser Pro Gly Leu
1 5 10 15
Phe Gin Ser Gly Gly Asp Asn Gly Leu Gly Gly His Asn Ala Asn Ser
                          20 25 30
Ala Leu Gly Gln Gln Pro Ile Asp Arg Gln Thr Ile Glu Gln Met Ala
                35 40 45
Gin Leu Leu Ala Glu Leu Leu Lys Ser Leu Leu Ser Pro Gln Ser Gly
        50 55 60
Asn Ala Ala Thr Gly Ala Gly Gly Asn Asp Gln Thr Thr Gly Val Gly
65 70 75 80
Asn Ala Gly Gly Leu Asn Gly Arg Lys Gly Thr Ala Gly Thr Thr Pro
                                85 90 95
Gln Ser Asp Ser Gln Asn Met Leu Ser Glu Met Gly Asn Asn Gly Leu
                        100 105 110
Asp Gln Ala Ile Thr Pro Asp Gly Gln Gly Gly Gly Gln Ile Gly Asp
                115 120 125
Asn Pro Leu Leu Lys Ala Met Leu Lys Leu Ile Ala Arg Met Met Asp
        130 135 140
Gly Gln Ser Asp Gln Phe Gly Gln Pro Gly Thr Gly Asn Asn Ser Ala
145 150 155 160
Ser Ser Gly Thr Ser Ser Ser Gly Gly Ser Pro Phe Asn Asp Leu Ser
                                165 170 175
Gly Gly Lys Ala Pro Ser Thr Asn Ser Pro Ser Gly Asn Tyr Ser Pro
                        180 185 190
Val Ser Thr Phe Ser Pro Pro Ser Thr Pro Thr Ser Pro Thr Ser Pro
                195 200 205
Leu Asp Phe Pro Ser Ser Pro Thr Lys Ala Ala Gly Gly Ser Thr Pro
        210 215 220
Val Thr Asp His Pro Asp Pro Val Gly Ser Ala Gly Ile Gly Ala Gly
225 230 235 240
Asn Ser Val Ala Phe Thr Ser Ala Gly Ala Asn Gln Thr Val Leu His
                                245 250 255
Asp Thr Ile Thr Val Lys Ala Gly Gln Val Phe Asp Gly Lys Gly Gln
                        260 265 270
Thr Phe Thr Ala Gly Ser Glu Leu Gly Asp Gly Gly Gln Ser Glu Asn
                275 280 285
Gln Lys Pro Leu Phe Ile Leu Glu Asp Gly Ala Ser Leu Lys Asn Val
        290 295 300
Thr Met Gly Asp Asp Gly Ala Asp Gly Ile His Leu Tyr Gly Asp Ala
305 310 315 320
Lys Ile Asp Asn Leu His Val Thr Asn Val Gly Glu Asp Ala Ile Thr
                                325 330 335
Val Lys Pro Asn Ser Ala Gly Lys Lys Ser His Val Glu Ile Thr Asn
                        340 345 350
Ser Ser Phe Glu His Ala Ser Asp Lys Ile Leu Gln Leu Asn Ala Asp
                355 360 365
Thr Asn Leu Ser Val Asp Asn Val Lys Ala Lys Asp Phe Gly Thr Phe
        370 375 380
Val Arg Thr Asn Gly Gly Gln Gln Gly Asn Trp Asp Leu Asn Leu Ser
385 390 395 400
His Ile Ser Ala Glu Asp Gly Lys Phe Ser Phe Val Lys Ser Asp Ser
                                405 410 415
Glu Gly Leu Asn Val Asn Thr Ser Asp Ile Ser Leu Gly Asp Val Glu
                        420 425 430
Asn His Tyr Lys Val Pro Met Ser Ala Asn Leu Lys Val Ala Glu
                435 440 445
```

This protein or polypeptide is acidic, rich in glycine and serine, and lacks cysteine. It is also heat stable, protease sensitive, and suppressed by inhibitors of plant metabolism. The protein or polypeptide of the present invention has a predicted molecular size of ca. 4.5 kDa.

This hypersensitive response elicitor from Erwinia amylovora has 2 hypersensitive response eliciting domains. The first domain extends, within SEQ. ID. No. 6, from amino acid 5 to amino acid 64, particularly from amino acid 31 to amino acid 57. The acidic unit in the first domain extends, within SEQ. ID. No. 6, from amino acid 5 to amino acid 45, particularly from amino acid 31 to amino acid 45. The alpha-helix in the first domain extends, within SEQ. ID. No. 6, from amino acid 45 to amino acid 64, particularly from amino acid 45 to amino acid 64. The second domain extends, within SEQ. ID. No. 6, from amino acid 103 to amino acid 146, particularly from amino acid 116 to amino acid 140. The acidic unit in the second domain extends, within SEQ. ID. No. 6, from amino acid 103 to amino acid 131, particularly from amino acid 116 to amino acid 131. The alpha-helix in the second domain extends, within SEQ. ID. No. 6, from amino acid 131 to amino acid 146, particularly from amino acid 131 to amino acid 140.

Another potentially suitable hypersensitive response elicitor from Erwinia amylovora is disclosed in U.S. patent application Ser. No. 09/120,663, which is hereby incorporated by reference. The protein is encoded by a DNA molecule having a nucleic acid sequence of SEQ. ID. No. 7 as follows:

| | |
|---|---|
| ATGGAATTAA AATCACTGGG AACTGAACAC AAGGCGGCAG TACACACAGC GGCGCACAAC | 60 |
| CCTGTGGGGC ATGGTGTTGC CTTACAGCAG GGCAGCAGCA GCAGCAGCCC GCAAAATGCC | 120 |
| GCTGCATCAT TGGCGGCAGA AGGCAAAAAT CGTGGGAAAA TGCCGAGAAT TCACCAGCCA | 180 |
| TCTACTGCGG CTGATGGTAT CAGCGCTGCT CACCAGCAAA AGAAATCCTT CAGTCTCAGG | 240 |
| GGCTGTTTGG GGACGAAAAA ATTTTCCAGA TCGGCACCGC AGGGCCAGCC AGGTACCACC | 300 |
| CACAGCAAAG GGGCAACATT GCGCGATCTG CTGGCCCGGG ACGACGGCGA AACGCAGCAT | 360 |
| GAGGCGGCCG CGCCAGATGC GGCGCGTTTG ACCCGTTCGG GCGGCGTCAA ACGCCGCAAT | 420 |
| ATGGACGACA TGGCCGGGCG GCCAATGGTG AAAGGTGGCA GCGGCGAAGA TAAGGTACCA | 480 |
| ACGCAGCAAA AACGGCATCA GCTGAACAAT TTTGGCCAGA TGCGCCAAAC GATGTTGAGC | 540 |
| AAAATCGCTC ACCCGGCTTC AGCCAACGCC GGCGATCGCC TGCAGCATTC ACCGCCGCAC | 600 |
| ATCCCGGGTA GCCACCACGA AATCAAGGAA GAACCGGTTG GCTCCACCAG CAAGGCAACA | 660 |
| ACGGCCCACG CAGACAGAGT GGAAATCGCT CAGGAAGATG ACGACAGCGA ATTCCAGCAA | 720 |
| CTGCATCAAC AGCGCCTGGC GCGCGAACGG GAAAATCCAC CGCAGCCGCC CAAACTCGGC | 780 |
| GTTGCCACAC CGATTAGCGC CAGGTTTCAG CCCAAACTGA CTGCGGTTGC GGAAAGCGTC | 840 |
| CTTCAGGGGA CAGATACCAC GCAGTCACCC CTTAAGCCGC AATCAATGCT GAAAGGAAGT | 900 |
| GGAGCCGGGG TAACGCCGCT GGCGGTAACG CTGGATAAAG GCAAGTTGCA GCTGGCACCG | 960 |
| GATAATCCAC CCGCGCTCAA TACGTTGTTG AAGCAGACAT TGGGTAAAGA CACCCAGCAC | 1020 |
| TATCTGGCGC ACCATGCCAG CAGCGACGGT AGCCAGCATC TGCTGCTGGA CAACAAACGC | 1080 |
| CACCTGTTTG ATATCAAAAG CACCGCCACC AGCTATAGCG TGCTGCACAA CAGCCACCCC | 1140 |
| GGTGAGATAA AGGGCAAGCT GGCGCAGGCG GGTACTGGCT CCGTCAGCGT AGACGGTAAA | 1200 |
| AGCGGCAAGA TCTCGCTGGG GAGCGGTACG CAAAGTCACA ACAAAACAAT GCTAAGCCAA | 1260 |
| CCGGGGGAAG CGCACCGTTC CTTATTAACC GGCATTTGGC AGCATCCTGC TGGCGCAGCG | 1320 |
| CGGCCGCAGG GCGAGTCAAT CCGCCTGCAT GACGACAAAA TTCATATCCT GCATCCGGAG | 1380 |
| CTGGGCGTAT GGCAATCTGC GGATAAAGAT ACCCACAGCC AGCTGTCTCG CCAGGCAGAC | 1440 |
| GGTAAGCTCT ATGCGCTGAA AGACAACCGT ACCCTGCAAA ACCTCTCCGA TAATAAATCC | 1500 |
| TCAGAAAAGC TGGTCGATAA AATCAAATCG TATTCCGTTG ATCAGCGGGG GCAGGTGGCG | 1560 |
| ATCCTGACGG ATACTCCCGG CCGCCATAAG ATGAGTATTA TGCCCTCGCT GGATGCTTCC | 1620 |
| CCGGAGAGCC ATATTTCCCT CAGCCTGCAT TTTGCCGATG CCCACCAGGG GTTATTGCAC | 1680 |
| GGGAAGTCGG AGCTTGACGC ACAATCTGTC GCGATCAGCC ATGGGCGACT GGTTGTGGCC | 1740 |
| CATAGCGAAG GCAAGCTGTT TAGCGCCGCC ATTCCGAAGC AAGGGGATGG AAACGAACTG | 1800 |
| AAAATGAAAG CCATGCCTCA GCATGCGCTC GATGAACATT TTGGTCATGA CCACCAGATT | 1860 |
| TCTGGATTTT TCCATGACGA CCACGCCCAG CTTAATGCGC TGGTGAAAAA TAACTTCAGG | 1920 |
| CAGCAGCATG CCTGCCCGTT GGGTAACGAT CATCAGTTTC ACCCCGGCTG GAACCTGACT | 1980 |
| GATGCGCTGG TTATCGACAA TCAGCTGGGG CTGCATCATA CCAATCCTGA ACCGCATGAG | 2040 |
| ATTCTTGATA TGGGGCATTT AGGCAGCCTG GCGTTACAGG AGGGCAAGCT TCACTATTTT | 2100 |
| GACCAGCTGA CCAAAGGGTG GACTGGCGCG GAGTCAGATT GTAAGCAGCT GAAAAAAGGC | 2160 |
| CTGGATGGAG CAGCTTATCT ACTGAAAGAC GGTGAAGTGA AACGCCTGAA TATTAATCAG | 2220 |
| AGCACCTCCT CTATCAAGCA CGGAACGGAA AACGTTTTTT CGCTGCCGCA TGTGCGCAAT | 2280 |
| AAACCGGAGC CGGGAGATGC CCTGCAAGGG CTGAATAAAG ACGATAAGGC CCAGGCCATG | 2340 |
| CCGGTGATTG GGGTAAATAA ATACCTGGCG CTGACGGAAA AAGGGGACAT TCGCTCCTTC | 2400 |
| CAGATAAAAC CCGGCACCCA GCAGTTGGAG CGGCCGGCAC AAACTCTCAG CCGCGAAGGT | 2460 |
| ATCAGCGGCG AAC | |
| GAAAGA CATECATGTC GACCACAAGC AGAACCTGTA TGCCTTGACC | 2520 |
| CACGAGGGAG AGGTGTTTCA TCAGCCGCGT GAAGCCTGGC AGAATGGTGC CGAAAGCAGC | 2580 |
| AGCTGGCACA AACTGGCGTT GCCACAGAGT GAAAGTAAGC TAAAAAGTCT GGACATGAGC | 2640 |
| CATGAGCACA AACCGATTGC CACCTTTGAA GACGGTAGCC AGCATCAGCT GAAGGCTGGC | 2700 |
| GGCTGGCACG CCTATGCGGC ACCTGAACGC GGGCCGCTGG CGGTGGGTAC CAGCGGTTCA | 2760 |
| CAAACCGTCT TTAACCGACT AATGCAGCGG GTGAAAGGCA AGGTGATCCC AGGCAGCGGG | 2820 |
| TTGACGGTTA AGCTCTCGGC TCAGACGGGG GGAATGACCG GCGCCGAAGG GCGCAAGGTC | 2880 |
| AGCAGTAAAT TTTCCGAAAG GATCCGCGCC TATGCGTTCA ACCCAACAAT GTCCACGCCG | 2940 |
| CGACCGATTA AAAATGCTGC TTATGCCACA CAGCACGGCT GGCAGCGGCG TGAGGGGTTG | 3000 |
| AAGCCGTTGT ACGAGATGCA GGGAGCGCTG ATTAAACAAC TGGATGCGCA TAACGTTCGT | 3060 |
| CATAACGCGC CACAGCCAGA TTTGCAGAGC AAACTGGAAA CTCTGGATTT AGGCGAACAT | 3120 |
| GGCGCAGAAT TGCTTAACGA CATGAAGCGC TTCCGCGACG AACTGGAGCA GAGTGCAACC | 3180 |
| CGTTCGGTGA CCGTTTTAGG TCAACATCAG GGAGTGCTAA AAAGCAACGG TGAAATCAAT | 2240 |
| AGCGAATTTA AGCCATCGCC CGGCAAGGCG TTGGTCCAGA GCTTTAACGT CAATCGCTCT | 3300 |
| GGTCAGGATC TAAGCAAGTC ACTGCAACAG GCAGTACATG CCACGCCGCC ATCCGCAGAG | 3360 |
| AGTAAACTGC AATCCATGCT GGGGCACTTT CTCAGTGCCG GGGTGGATAT GAGTCATCAG | 3420 |
| AAGGGCGAGA TCCCGCTGGG CCGCCAGCGC GATCCGAATG ATAAAACCGC ACTGACCAAA | 3480 |
| TCCCGTTTAA TTTTAGATAC CGTGACCATC GGTGAACTGC ATGAACTGGC CGATAAGGCG | 3540 |
| AAACTGGTAT CTGACCATAA ACCCGATGCC GATCAGATAA AACAGCTGCG CCAGCAGTTC | 3600 |
| GATACGCTGC GTGAAAAGCG GTATGAGAGC AATCCGGTGA AGCATTACAC CGATATGGGC | 3660 |
| TTCACCCATA ATAAGGCGCT GGAAGCAAAC TATGATGCGG TCAAAGCCTT TATCAATGCC | 3720 |
| TTTAAGAAAG AGCACCACGG CGTCAATCTG ACCACGCGTA CCGTACTGCA ATCACAGGGC | 3780 |
| AGTGCGGAGC TGGCGAAGAA GCTCAAGAAT ACGCTGTTGT CCCTGGACAG TGGTGAAAGT | 3840 |
| ATGAGCTTCA GCCGGTCATA TGGCGGGGGC GTCAGCACTG TCTTTGTGCC TACCCTTAGC | 3900 |
| AAGAAGGTGC CAGTTCCCGT GATCCCCGGA GCCGGCATCA CGCTGGATCG CGCCTATAAC | 3960 |
| CTGAGCTTCA GTCGTACCAG CGGCGGATTG AACGTCAGTT TTGGCCGCGA CGGCGGGGTG | 4020 |
| AGTGGTAACA TCATGGTCGC TACCGGCCAT GATGTGATGC CCTATATGAC CGGTAAGAAA | 4080 |
| ACCAGTGCAG GTAACGCCAG TGACTGGTTG AGCGCAAAAC ATAAAATCAG CCCGGACTTG | 4140 |
| CGTATCGGCG CTGCTGTGAG TGGCACCCTG CAAGGAACGC TACAAAACAG CCTGAAGTTT | 4200 |
| AAGCTGACAG AGGATGAGCT GCCTGGCTTT ATCCATGGCT TGACGCATGG CACGTTGACC | 4260 |
| CCGGCAGAAC TGTTGCAAAA GGGCATCGAA CATCAGATGA AGCAGGGCAG CAAACTGACG | 4320 |
| TTTAGCGTCG ATACCTCGGC AAATCTGGAT CTGCGTGCCG GTATCAATCT GAACGAAGAC | 4380 |
| GGCAGTAAAC CAAATGGTGT CACTGCCCGT GTTTCTGCCG GGCTAAGTGC ATCGGCAAAC | 4440 |
| CTCGCCGCCG GCTCGCGTGA ACGCAGCACC ACCTCTGGCC AGTTTGGCAG CACGACTTCG | 4500 |
| GCCAGCAATA ACCGCCCAAC CTTCCTCAAC GGGGTCGGCG CGGGTGCTAA CCTGACGGCT | 4560 |
| GCTTTAGGGG TTGCCCATTC ATCTACGCAT GAAGGGAAAC CGGTCGGGAT CTTCCCGGCA | 4620 |
| TTTACCTCGA CCAATGTTTC GGCAGCGCTG GCGCTGGATA ACCGTACCTC ACAGAGTATC | 4680 |
| AGCCTGGAAT TGAAGCGCGC GGAGCCGGTG ACCAGCAACG ATATCAGCGA GTTGACCTCC | 4740 |
| ACGCTGGGAA AACACTTTAA GGATAGCGCC ACAACGAAGA TGCTTGCCGC TCTCAAAGAG | 4800 |
| TTAGATGACG CTAAGCCCGC TGAACAACTG CATATTTTAC AGCAGCATTT CAGTGCAAPA | 4860 |
| GATGTCGTCG GTGATCAACG CTACGAGGCG GTGCGCAACC TGAAAAAACT GGTGATACGT | 4920 |
| CAACAGGCTG CGGACAGCCA CAGCATCGAA TTAGGATCTG CCAGTCACAG CACGACCTAC | 4980 |
| AATAATCTGT CGAGAATAAA TAATGACGGC ATTGTCGAGC TGCTACACAA ACATTTCGAT | 5040 |
| GCGGCATTAC CAGCAAGCAG TGCCAAACGT CTTGGTGAAA TGATGAATAA CCATCCCGCA | 5100 |
| CTGAAAGATA TTATTAAGCA GCTGCAAAGT ACGCCGTTCA GCAGCGCCAG CGTGTCGATG | 5160 |
| GAGCTGAAAG ATGGTCTGCG TGAGCAGACG GAAAAAGCAA TACTGGACGG TAAGGTCGGT | 5220 |
| CGTGAAGAAG TGGGAGTACT TTTCCAGGAT CGTAACAACT TGCGTGTTAA ATCGGTCAGC | 5280 |
| GTCAGTCAGT CCGTCAGCAA AAGCGAAGGC TTCAATACCC CAGCGCTGTT ACTGGGGACG | 5340 |
| AGCAACAGCG CTGCTATGAG CATGGAGCGC AACATCGGAA CCATTAATTT TAAATACGGC | 5400 |
| CAGGATCAGA ACACCCCACG GCGATTTACC CTGGAGGGTG GAATAGCTCA GGCTAATCCG | 5460 |
| CAGGTCGCAT CTGCGCTTAC TGATTTGAAG AAGGAAGGGC TGGAAATGAA GAGCTAA | 5517 |

This DNA molecule is known as the dspE gene for Erwinia amylovora. This isolated DNA molecule of the present invention encodes a protein or polypeptide which elicits a plant pathogen's hypersensitive response having an amino acid sequence of SEQ. ID. No. 8 as follows:

```
Met Glu Leu Lys Ser Leu Gly Thr Glu His Lys Ala Ala Val His Thr
1 5 10 15
Ala Ala His Asn Pro Val Gly His Gly Val Ala Leu Gln Gln Gly Ser
                        20 25 30
Ser Ser Ser Ser Pro Gln Asn Ala Ala Ala Ser Leu Ala Ala Glu Gly
                35 40 45
Lys Asn Arg Gly Lys Met Pro Arg Ile His Gln Pro Ser Thr Ala Ala
        50 55 60
Asp Gly Ile Ser Ala Ala His Gln Gln Lys Lys Ser Phe Ser Leu Arg
65 70 75 80
Gly Cys Leu Gly Thr Lys Lys Phe Ser Arg Ser Ala Pro Gln Gly Gln
                                85 90 95
Pro Gly Thr Thr His Ser Lys Gly Ala Thr Leu Arg Asp Leu Leu Ala
                        100 105 110
Arg Asp Asp Gly Glu Thr Gln His Glu Ala Ala Ala Pro Asp Ala Ala
                115 120 125
Arg Leu Thr Arg Ser Gly Gly Val Lys Arg Arg Asn Met Asp Asp Met
        130 135 140
Ala Gly Arg Pro Met Val Lys Gly Gly Ser Gly Glu Asp Lys Val Pro
145 150 155 160
Thr Gln Gln Lys Arg His Gln Leu Asn Asn Phe Gly Gln Met Arg Gln
                                165 170 175
Thr Met Leu Ser Lys Met Ala His Pro Ala Ser Ala Asn Ala Gly Asp
                        180 185 190
Arg Leu Gln His Ser Pro Pro His Ile Pro Gly Ser His His Glu Ile
                195 200 205
Lys Glu Glu Pro Val Gly Ser Thr Ser Lys Ala Thr Thr Ala His Ala
        210 215 220
Asp Arg Val Glu Ile Ala Gln Glu Asp Asp Asp Ser Glu phe Gln Gln
225 230 235 240
Leu His Gln Gln Arg Leu Ala Arg Glu Arg Glu Asn Pro Pro Gln Pro
                                245 250 255
Pro Lys Leu Gly Val Ala Thr Pro Ile Ser Ala Arg Phe Gln Pro Lys
                        260 265 270
Leu Thr Ala Val Ala Glu Ser Val leu Glu Gly Thr Asp Thr Thr Gln
                275 280 285
Ser Pro Leu Lys Pro Gln Ser Met Leu Lys Gly Ser Gly Ala Gly Val
        290 295 300
Thr Pro Leu Ala Val Thr Leu Asp lys Gly Lys Leu Gln Leu Ala Pro
305 310 315 320
Asp Asn Pro Pro Ala Leu Asn Thr Leu Leu Lys Gln Thr Leu Gly Lys
                                325 330 335
Asp Thr Gln His Tyr Leu Ala His His Ala Ser Ser Asp Gly Ser Gln
                        340 345 350
His Leu Leu Leu Asp Asn Lys Gly His Leu Phe Asp Ile Lys Ser Thr
                355 360 365
Ala Thr Ser Tyr Ser Val Leu His Asn Ser His Pro Gly Glu Ile Lys
        370 375 380
Gly Lys Leu Ala Gln Ala Gly Thr Gly Ser Val Ser Val Asp Gly Lys
385 390 395 400
Ser Gly Lys Ile Ser Leu Gly Ser Gly Thr Gln Ser His Asn Lys Thr
                                405 410 415
Met Leu Ser Gln Pro Gly Glu Ala His Arg Ser Leu Leu Thr Gly Ile
                        420 425 430
Trp Gln His Pro Ala Gly Ala Ala Arg Pro Gln Gly Glu Ser Ile Arg
                435 440 445
Leu His Asp Asp Lys Ile His Ile Leu His Pro Glu Leu Gly Val Trp
        450 455 460
Gln Ser Ala Asp Lys Asp Thr His Ser Gln Leu Ser Arg Gln Ala Asp
465 470 475 480
Gly Lys Leu Tyr Ala Leu Lys Asp Asn Arg Thr Leu Gln Asn Leu Ser
                                485 490 495
Asp Asn Lys Ser Ser Glu Lys Leu Val Asp Lys Ile Lys Ser Tyr Ser
                        500 505 510
Val Asp Gln Arg Gly Gln Val Ala Ile Leu Thr Asp Thr Pro Gly Arg
                515 520 525
His Lys Met Ser Ile Met Pro Ser Leu Asp Ala Ser Pro Glu Ser His
        530 535 540
Ile Ser Leu Ser Leu His Phe Ala Asp Ala His Gln Gly Leu Leu His
545 550 555 560
Gly Lys Ser Glu Leu Glu Ala Gln Ser Val Ala Ile Ser His Gly Arg
                                565 570 575
Leu Val Val Ala Asp Ser Glu Gly Lys Leu Phe Ser Ala Ala Ile Pro
                        580 585 590
Lys Gln Gly Asp Gly Asn Glu Leu Lys Met Lys Ala Met Pro Gln His
                595 600 605
Ala Leu Asp Glu His Phe Gly His Asp His Gln Ile Ser Gly Phe Phe
        610 615 620
His Asp Asp His Gly Gln Leu Asn Ala Leu Val Lys Asn Asn Phe Arg
625 630 635 640
Gln Gln His Ala Cys Pro Leu Gly Asn Asp His Gln Phe His Pro Gly
                                645 650 655
Trp Asn Leu Thr Asp Ala Leu Val Ile Asp Asn Gln Leu Gly Leu His
                        660 665 670
His Thr Asn Pro Glu Pro His Glu Ile Leu Asp Met Gly His Leu Gly
                675 680 685
Ser Leu Ala Leu Gln Glu Gly Lys Leu His Tyr Phe Asp Gln Leu Thr
        690 695 700
Lys Gly Trp Thr Gly Ala Glu Ser Asp Cys Lys Gln Leu Lys Lys Gly
705 710 715 720
Leu Asp Gly Ala Ala Tyr Leu Leu Lys Asp Gly Glu Val Lys Arg Leu
                                725 730 735
Asn Ile Asn Gln Ser Thr Ser Ser Ile Lys His Gly Thr Glu Asn Val
                        740 745 750
Phe Ser Leu pro His Val Arg Asn Lys Pro Glu Pro Gly Asp Ala leu
                755 760 765
Gln Gly Leu Asn Lys Asp Asp Lys Ala Gln Ala Met Ala Val Ile Gly
        770 775 780
Val Asn Lys Tyr Leu Ala Leu Thr Glu Lys Gly Asp Ile Arg Ser Phe
785 790 795 800
Gln Ile Lys Pro Gly Thr Gln Gln Leu Glu Arg Pro Ala Gln Thr Leu
                                805 810 815
Ser Arg Glu Gly Ile Ser Gly Glu Leu Lys Asp Ile His Val Asp His
                        820 825 830
Lys Gln Asn Leu Tyr Ala Leu Thr His Glu Gly Glu Val Phe his Gln
                835 840 845
Pro Arg Glu Ala Trp Gln Asn Gly Ala Glu Ser Ser Ser Trp His Lys
        850 855 860
Leu Ala Leu Pro Gln Ser Glu Ser Lys Leu Lys Ser Leu Asp Met Ser
865 870 875 880
His Glu His Lys Pro Ile Ala Thr Phe Glu Asp Gly Ser Gln His Gln
                                885 890 895
Lun Lys Ala Gly Gly Trp His Ala Tyr Ala Ala Pro Glu Arg Gly Pro
                        900 905 910
Leu Ala Val Gly Thr Her Gly Ser Gln Thr Val Phe Asn Arg Leu Met
                915 920 925
Gln Gly Val Lys Gly Lys Val Ile Pro Gly Her Gly Leu Thr Val Lys
        930 935 940
Leu Ser Ala Gln Thr Gly Gly Met Thr Gly Ala Glu Gly Ary Lys Val
945 950 955 960
Ser Her Lys Phe Ser Gln Arg Ile Arg Ala Tyr Ala Phe Asn Pro Thr
                                965 970 975
Met Ser Thr Pro Ary Pro Ile Lys Asn Ala Ala Tyr Ala Thr Gln His
                        980 985 990
Gly Trp Gln Gly Arg Gln Gly Leu Lys Pro Leu Tyr Glu Met Gln Gly
                995 1000 1005
Ala Leu Ile Lys Gln Leu Asp Ala His Asn Val Arg His Asn Ala Pro
        1010 1015 1020
Gln Pro Asp Leu Gln Ser Lys Leu Gln Thr Leu Asp Len Gly Gln His
1025 1030 1035 1040
Gly Ala Glu Leu Leu Asn Asp Met Lys Arg Phe Arg Asp Glu Leu Glu
                                1045 1050 1055
Gln Her Ala Thr Arg Ser Val Thr Val Leu Gly Gln His Gln Gly Val
                        1060 1065 1070
Leu Lys Ser Asn Gly Glu Ile Asn Ser Gln Phe Lys Pro Ser Pro Gly
                1075 1080 1085
Lys Ala Leu Val Gln Ser Phe Asn Val Asn Arg Ser Gly Gln Asp Leu
        1090 1095 1100
Ser Lys Her Leu Gln Gln Ala Val His Ala Thr Pro Pro Ser Ala Gln
1105 1110 1115 1120
Ser Lys Leu Gln Ser Met Leu Gly His Phe Val Her Ala Gly Val Asp
                                1125 1130 1135
Met Ser His Gln Lys Gly Gln Ile Pro Leu Gly Arg Gln Arg Asp Pro
                        1140 1145 1150
Asn Asp Lys Thr Ale Leu Thr Lys Ser Arg Leu Ile Leu Asp Thr Val
                1155 1160 1165
Thr Ile Gly Gln Leu His Glu Leu Ala Asp Lys Ala Lys Leu Val Ser
        1170 1175 1180
Asp His Lys Pro Asp Ala Asp Gln Ile Lys Gln Leu Arg Gln Gln Phe
1185 1190 1195 1200
Asp Thr Leu Arg Glu Lys Arg Tyr Gln Ser Asn Pro Val Lys His Tyr
                                1205 1210 1215
Thr Asp Met Gly Phe Thr His Asn Lys Ala Leu Gln Ala Asn Tyr Asp
                        1220 1225 1230
Ala Val Lys Ala Phe Ile Asn Ala Phe Lys Lys Gln His His Gly Val
                1235 1240 1245
Asn Leu Thr Thr Arg Thr Val Leu Glu Ser Gln Gly Ser Ala Glu Leu
        1250 1255 1260
Ala Lys Lys Leu Lys Asn Thr Leu Leu Ser Leu Asp Ser Gly Glu Ser
1265 1270 1275 1280
Met Ser Phe Ser Arg Ser Tyr Gly Gly Gly Val Ser Thr Val Phe Val
                                1285 1290 1295
Pro Thr Leu Ser Lys Lys Val Pro Val Pro Val Ile Pro Gly Ala Gly
                        1300 1305 1310
Ile Thr Leu Asp Arg Ala Tyr Asn Leu Ser Phe Ser Arg Thr Ser Gly
                1315 1320 1325
Gly Leu Asn Val Ser Phe Gly Arg Asp Gly Gly Val Ser Gly Asn Ile
        1330 1335 1340
Met Val Ala Thr Gly His Asp Val Met Pro Tyr Met Thr Gly Lys Lys
1345 1350 1355 1360
Thr Ser Ala Gly Asn Ala Ser Asp Trp Leu Ser Ala Lye His Lys Ile
                                1365 1370 1375
Ser Pro Asp Leu Arg Ile Gly Ala Ala Val Ser Gly Thr Leu Gln Gly
                        1380 1385 1390
Thr Leu Gln Asn Ser Leu Lys Phe Lys Leu Thr Glu Asp Glu Leu Pro
                1395 1400 1405
Gly Phe Ile His Gly Leu Thr His Gly Thr Leu Thr Pro Ala Glu Leu
        1410 1415 1420
Leu Gln Lys Gly Ile Glu His Gln Met Lys Gln Gly Ser Lys Leu Thr
1425 1430 1435 1440
Phe Ser Val Asp Thr Ser Ala Asn Leu Asp Leu Arg Ala Gly Ile Asn
                                1445 1450 1455
Leu Asn Glu Asp Gly Ser Lye Pro Asn Gly Val Thr Ala Arg Val Ser
                        1460 1465 1470
Ala Gly Leu Ser Ala Ser Ala Asn Leu Ala Ala Gly Ser Arg Glu Arg
                1475 1480 1485
Ser Thr Thr Ser Gly Gln Phe Gly Ser Thr Thr Ser Ala Ser Asn Asn
        1490 1495 1500
Arg Pro Thr Phe Leu Asn Gly Val Gly Ala Gly Ala Asn Leu Thr Ala
1505 1510 1515 1520
Ala Leu Gly Val Ala His Ser Ser Thr His Glu Gly Lye Pro Val Gly
                                1525 1530 1535
Ile Phe Pro Ala Phe Thr Ser Thr Asn Val Ser Ala Ala Leu Ala Leu
                        1540 1545 1550
Asp Asn Arg Thr Ser Gln Ser Ile Ser Leu Glu Leu Lys Ary Ala Glu
                1555 1560 1565
Pro Val Thr Ser Asn Asp Ile Ser Glu Leu Thr Ser Thr Leu Gly Lys
        1570 1575 1580
His Phe Lys Asp Ser Ala Thr Thr Lys Met Leu Ala Ala Leu Lys Glu
1585 1590 1595 1600
Leu Asp Asp Ala Lys Pro Ala Glu Gln Leu His Ile Leu Gln Gln His
                                1605 1610 1615
Phe Ser Ala Lys Asp Val Val Gly Asp Glu Arg Tyr Glu Ala Val Arg
                        1620 1625 1630
Asn Leu Lys Lys Leu Val Ile Arg Gln Gln Ala Ala Asp Ser His Ser
                1635 1640 1645
Met Glu Leu Gly Ser Ala Ser His Ser Thr Thr Tyr Asn Asn Leu Ser
        1650 1655 1660
Arg Ile Asn Asn Asp Gly Ile Val Glu Leu Leu His Lys His Phe Asp
1665 1670 1675 1680
Ala Ala Leu Pro Ala Ser Ser Ala Lys Arg Leu Gly Glu Met Met Asn
                                1685 1690 1695
Asn Asp Pro Ala Leu Lys Asp Ile Ile Lys Gln Leu Gln Ser Thr Pro
                        1700 1705 1710
Phe Ser Ser Ala Ser Val Ser Met Glu Leu Lys Asp Gly Leu Arg Glu
                1715 1720 1725
Gln Thr Glu Lys Ala Ile Leu Asp Gly Lys Val Gly Arg Glu Glu Val
        1730 1735 1740
Gly Val Leu Phe Gln Asp Arg Asn Asn Leu Arg Val Lys Ser Val Ser
1745 1750 1755 1760
Val Ser Gln Ser Val Ser Lys Ser Glu Gly Phe Aso Thr Pro Ala Leu
                                1765 1770 1775
Leu Leu Gly Thr Ser Asn Ser Ala Ala Met Ser Met Glu Arg Asn Ile
                        1780 1785 1790
Gly Thr Ile Asn Phe Lys Tyr Gly Gln Asp Gln Asn Thr Pro Arg Arg
                1795 1800 1805
Phe Thr Leu Glu Gly Gly Ile Ala Gln Ala Asn Pro Gln Val Ala Ser
        1810 1815 1820
Ala Leu Thr Asp Leu Lys Lys Glu Gly Leu Glu Met Lys Ser
1825 1830 1835
```

This protein or polypeptide is about 198 kDa and has a pl of 8.98.

The present invention relates to an isolated DNA molecule having a nucleotide sequence of SEQ. ID. No. 9 as follows:

| | |
|---|---|
| ATGACATCGT CACAGCAGCG GGTTGAAAGG TTTTTACAGT ATTTCTCCGC CCGCTCTAAA | 60 |
| ACGCCCATAC ATCTGAAAGA CCGGGTGTGC GCCCTGTATA ACGAACAAGA TGAGGAGGCG | 120 |
| GCGGTGCTGG AAGTACCGCA ACACAGCGAC AGCCTGTTAC TACACTGCCG AATCATTGAG | 180 |
| GCTGACCCAC AAACTTCAAT AACCCTGTAT TCGATGCTAT TACAGCTGAA TTTTGAAATG | 240 |
| GCGGCCATGC GCGGCTGTTG GCTGGCGCTG GATGAACTGC ACAACGTGCG TTTATGTTTT | 300 |
| CAGCAGTCGC TGGAGCATCT GGATGAAGCA AGTTTTAGCG ATATCGTTAG CGGCTTCATC | 360 |
| GAACATGCGG CAGAAGTGCG TGAGTATATA GCGCAATTAG ACGAGAGTAG CGCGGCATAA | 420 |

This is known as the dspF gene. This isolated DNA molecule of the present invention encodes a hypersensitive response elicitor protein or polypeptide having an amino acid sequence of SEQ. ID. No. 10 as follows:

```
Met Thr Ser Ser Gln Gln Arg Val Glu Arg Phe Leu Gln Tyr Phe Ser
1 5 10 15
Ala Gly Cys Lys Thr Pro Ile His Leu Lys Asp Gly Val Cys Ala Leu
                        20 25 30
Tyr Asn Glu Gln Asp Glu Glu Ala Ala Val Leu Glu Val Pro Gln His
                35 40 45
Ser Asp Ser Leu Leu Leu His Cys Arg Ile Ile Glu Ala Asp Pro Gln
        50 55 60
Thr Ser Ile Thr Leu Tyr Ser Met Leu Leu Gln Leu Asn Phe Glu Met
65 70 75 80
Ala Ala Met Arg Gly Cys Trp Leu Ala Leu Asp Glu Leu His Asn Val
                                85 90 95
Arg Leu Cys Phe Gln Gln Ser Leu Glu His Leu Asp Glu Ala Ser Phe
                        100 105 110
Ser Asp Ile Val Ser Gly Phe Ile Glu His Ala Ala Glu Val Arg Glu
                115 120 125
Tyr Ile Ala Gln Leu Asp Glu Ser Ser Ala Ala
        130 135
```

This protein or polypeptide is about 16 kDa and has a pI of 4.45.

The hypersensitive response elicitor polypeptide or protein derived from Pseudomonas syringae has an amino acid sequence corresponding to SEQ. ID. No. 11 as follows:

```
Met Gln Ser Leu Ser Leu Asn Ser Ser Ser Leu Gln Thr Pro Ala Met
1 5 10 15
Ala Leu Val Leu Val Arg Pro Glu Ala Glu Thr Thr Gly Ser Thr Ser
                        20 25 30
Ser Lys Ala Leu Gln Glu Val Val Val Lys Leu Ala Glu Glu Leu Met
                35 40 45
Arg Asn Gly Gln Leu Asp Asp Ser Ser Pro Leu Gly Lys Leu Leu Ala
        50 55 60
Lys Ser Met Ala Ala Asp Gly Lys Ala Gly Gly Gly Ile Glu Asp Val
65 70 75 80
Ile Ala Ala Leu Asp Lys Leu Ile His Glu Lys Leu Gly Asp Asn Phe
                                85 90 95
Gly Ala Ser Ala Asp Ser Ala Ser Gly Thr Gly Gln Gln Asp Leu Met
                        100 105 110
Thr Gln Val Leu Asn Gly Leu Ala Lys Ser Met Leu Asp Asp Leu Leu
                115 120 125
Thr Lys Gln Asp Gly Gly Thr Ser Phe Ser Glu Asp Asp Met Pro Met
        130 135 140
Leu Asn Lys Ile Ala Gln Phe Met Asp Asp Asn Pro Ala Gln Phe Pro
145 150 155 160
Lys Pro Asp Ser Gly Ser Trp Val Asn Glu Leu Lys Gla Asp Asn Phe
                                165 170 175
Leu Asp Gly Asp Glu Thr Ala Ala Phe Arg Ser Ala Leu Asp Ile Ile
                        180 185 190
Gly Gln Gln Leu Gly Asn Gln Gln Ser Asp Ala Gly Ser Leu Ala Gly
                195 200 205
Thr Gly Gly Gly Leu Gly Thr Pro Ser Ser Phe Ser Asn Asn Ser Ser
        210 215 220
Val Met Gly Asp Pro Leu Ile Asp Ala Asn Thr Gly Pro Gly Asp Ser
225 230 235 240
Gly Asn Thr Arg Gly Glu Ala Gly Gln Leu Ile Gly Glu Leu Ile Asp
                                245 250 255
Arg Gly Leu Gln Ser Val Leu Ala Gly Gly Gly Leu Gly Thr Pro Val
                        260 265 270
Asn Thr Pro Gln Thr Gly Thr Ser Ala Asn Gly Gly Gln Ser Ala Gln
                275 280 285
Asp Leu Asp Gln Leu Leu Gly Gly Leu Leu Leu Lys Gly Leu Glu Ala
        290 295 300
Thr Leu Lys Asp Ala Gly Gln Thr Gly Thr Asp Val Gln Ser Ser Ala
305 310 315 320
Ala Gln Ile Ala Thr Leu Leu Val Ser Thr Leu Leu Gln Gly Thr Arg
                                325 330 335
Asn Gln Ala Ala Ala
                        340
```

This hypersensitive response elicitor polypeptide or protein has a molecular weight of 34-35 kDa. It is rich in glycine (about 13.5%) and lacks cysteine and tyrosine. Further information about the hypersensitive response elicitor derived from Pseudomonas syringae is found in He, S. Y., H. C. Huang, and A. Collmer, “Pseudomonas syringae pv. syringae Harpin_{Pss}: a Protein that is Secreted via the Hrp Pathway and Elicits the Hypersensitive Response in Plants,” Cell 73:1255-1266 (1993), which is hereby incorporated by reference. The DNA molecule encoding the hypersensitive response elicitor from Pseudomonas syringae has a nucleotide sequence corresponding to SEQ. ID. No. 12 as follows:

| | |
|---|---|
| ATGCAGAGTC TCAGTCTTAA CAGCAGCTCG CTGCAAACCC CGGCAATGGC CCTTGTCCTG | 60 |
| GTACGTCCTG AAGCCGAGAC GACTGGCAGT ACGTCGAGCA AGGCGCTTCA GGAAOTTGTC | 120 |
| GTGAAGCTGG CCGAGGAACT GATGCGCAAT GGTCAACTCG ACGACAGCTC GCCATTGGGA | 180 |
| AAACTGTTGG CCAAGTCGAT GGCCGCAGAT GGCAAGGCGG GCGGCGGTAT TGAGGATGTC | 240 |
| ATCGCTGCGC TGGACAAGCT GATCCATGAA AAGCTCGGTG ACAACTTCGG CGCGTCTGCG | 300 |
| GACAGCGCCT CGGGTACCGG ACAGCAGGAC CTGATGACTC AGGTGCTCAG TGGCCTGGCC | 360 |
| AAGTCGATGC TCGATGATCT TCTGACCAAG CAGGATGGCG GGACAAGCTT CTCCGAAGAC | 420 |
| GATATGCCGA TGCTGAACAA GATCGCGCAG TTCATGGATG ACAATCCCGC ACAGTTTCCC | 480 |
| AAGCCGGACT CGGGCTCCTG GGTGAACGAA CTCAAGGAAG ACAACTTCCT TGATGGCGAC | 540 |
| GAAACGGCTG CGTTCCGTTC GGCACTCGAC ATCATTGGCC AGCAACTGGG TAATCAGCAG | 600 |
| AGTGACGCTG GCAGTCTGGC AGGGACGGGT GGAGGTCTGG GCACTCCGAG CAGTTTTTCC | 660 |
| AACAACTCGT CCGTGATGGG TGATCCGCTG ATCGACGCCA ATACCGGTCC CGGTGACAGC | 720 |
| GGCAATACCC GTGGTGAAGC GGGGCAACTG ATCGGCGAGC TTATCGACCG TGGCCTGCAA | 780 |
| TCGGTATTGG CCGGTGGTGG ACTGGGCACA CCCGTAAACA CCCCGCAGAC CGGTACGTCG | 840 |
| GCGAATGGCG GACAGTCCGC TCAGGATCTT GATCAGTTGC TGGGCGGCTT GCTGCTCAAG | 900 |
| GGCCTGGAGG CAACGCTCAA GGATGCCGGG CAAACAGGCA CCGACGTGCA GTCGAGCGCT | 960 |
| GCGCAAATCG CCACCTTGCT GGTCAGTACG CTGCTGCAAG GCACCCGCAA TCAGGCTGCA | 1020 |
| GCCTGA | 1026 |

Another potentially suitable hypersensitive response elicitor from Pseudomonas syringae is disclosed in U.S. patent application Ser. No. 09/120,817, which is hereby incorporated by reference. The protein has a nucleotide sequence of SEQ. ID. No. 13 as follows:

| | |
|---|---|
| TCCACTTCGC TGATTTTGAA ATTGGCAGAT TCATAGAAAC GTTCAGGTGT GGAAATCAGG | 60 |
| CTGAGTGCGC AGATTTCGTT GATAAGGGTG TGGTACTGGT CATTGTTGGT CATTTCAAGG | 120 |
| CCTCTGAGTG CGGTGCGGAG CAATACCAGT CTTCCTGCTG GCGTGTGCAC ACTGAGTCGC | 180 |
| AGGCATAGGC ATTTCAGTTC CTTGCGTTCG TTGGGCATAT AAAAAAAGGA ACTTTTAATA | 240 |
| ACAGTGCAAT GAGATGCCGG CAAAACGGGA ACCGGTCGCT GCCCTTTGCC ACTCACTTCG | 300 |
| AGCAAGCTCA ACCCCAAACA TCCACATCCC TATCGAACGG ACAGCGATAC GGCCACTTGC | 360 |
| TCTGGTAAAC CCTGGAGCTG GCGTCGGTCC AATTGCCCAC TTAGCGAGGT AACGCAGCAT | 420 |
| GAGCATCGGC ATCACACCCC GGCCGCAACA GACCACCACG CCACTCGATT TTTCGGCGCT | 480 |
| AAGCGGCAAG AGTCCTCAAC CAAACACGTT CGGCGAGCAG AACACTCAGC AAGCGATCGA | 540 |
| CCCGAGTGCA CTGTTGTTCG GCAGCGACAC ACAGAAAGAC GTCAACTTCG GCACGCCCGA | 600 |
| CAGCACCGTC CAGAATCCGC AGGACGCCAG CAAGCCCAAC GACAGCCAGT CCAACATCGC | 660 |
| TAAATTGATC AGTGCATTGA TCATGTCGTT GCTGCAGATG CTCACCAACT CCAATAAAAA | 720 |
| GCAGGACACC AATCAGGAAC AGCCTGATAG CCAGGCTCCT TTCCAGAACA ACGGCGGGCT | 780 |
| CGGTACACCG TCCGCCGATA GCGGGCGCGG CGGTACACCG GATGCGACAG GTGGCGGCGG | 840 |
| CGGTGATACG CCAAGCGCAA CAGGCGGTGG CGGCGGTGAT ACTCCGACCG CAACAGGCGG | 900 |
| TGGCGGCAGC GGTGOCGGCG GCACACCCAC TGCAACAGGT GGCGGCAGCG GTGGCACACC | 960 |
| CACTGCAACA GGCGGTGGCG AGGGTGGCGT AACACCGCAA ATCACTCCGC AGTTGGCCAA | 1020 |
| CCCTAACCGT ACCTCAGGTA CTGGCTCGGT GTCGGACACC CCAGGTTCTA CCGAGCAAGC | 1080 |
| CGGCAAGATC AATGTGGTGA AAGACACCAT CAAGGTCGGC GCTGGCGAAG TCTTTGACGG | 1140 |
| CCACGGCGCA ACCTTCACTG CCGACAAATC TATGGGTAAC GGAGACCAGG GCGAAAATCA | 1200 |
| GAAGCCCATG TTCGAGCTGG CTQAAGGCGC TACGTTGAAG AATGTGAACC TGGGTGAGAA | 1260 |
| CGAGGTCGAT GGCATCCACG TGAAAGCCAA AAACGCTCAG GAAGTCACCA TTGACAACGT | 1320 |
| GCATGCCCAG AACGTCGGTG AAGACCTGAT TACGGTCAAA GGCGAGGGAG GCGCAGCGGT | 138 0 |
| CACTAATCTG AACATCAAGA ACAGCAGTGC CAAAGGTGCA GACGACAAGG TTGTCCAGCT | 1440 |
| CAACGCCAAC ACTCACTTCA AAATCGACAA CTTCAAGGCC GACGATTTCG GCACGATGGT | 1500 |
| TCGCACCAAC GGTGGCAAGC AGTTTGATGA CATGAGCATC GAGCTGAACG GCATCGAAGC | 1560 |
| TAACCACGGC AAGTTCGCCC TGGTGAAAAG CGACAGTGAC GATCTGAAGC TGGCAACGGG | 1620 |
| CAACATCGCC ATGACCGACG TCAAACACGC CTACGATAAA ACCCAGGCAT CGACCCAACA | 1680 |
| CACCGAGCTT TGAATCCAGA CAAGTAGCTT GAAAAAAGGC GGTGGACTC | 1729 |

This DNA molecule is known as the dspE gene for Pseudomonas syringae. This isolated DNA molecule of the present invention encodes a protein or polypeptide which elicits a plant pathogen's hypersensitive response having an amino acid sequence of SEQ. ID. No. 14 as follows:

```
Met Ser Ile Gly Ile Thr Pro Arg Pro Gln Gln Thr Thr Thr Pro Leu
1 5 10 15
Asp Phe Ser Ala Leu Ser Gly Lys Ser Pro Gln Pro Asn Thr Phe Gly
                        20 25 30
Glu Gln Asn Thr Gln Gln Ala Ile Asp Pro Ser Ala Leu Leu Phe Gly
                35 40 45
Ser Asp Thr Gln Lys Asp Val Asn Phe Gly Thr Pro Asp Ser Thr Val
        50 55 60
Gln Asn Pro Gln Asp Ala Ser Lys Pro Asn Asp Ser Gln Ser Asn Ile
65 70 75 80
Ala Lys Leu Ile Ser Ala Leu Ile Met Ser Leu Leu Gln Met Leu Thr
                                85 90 95
Asn Ser Asn Lys Lys Gln Asp Thr Asn Gln Glu Gln Pro Asp Ser Gln
                        100 105 110
Ala Pro Phe Gln Asn Asn Gly Gly Leu Gly Thr Pro Ser Ala Asp Ser
                115 120 125
Gly Gly Gly Gly Thr Pro Asp Ala Thr Gly Gly Gly Gly Gly Asp Thr
        130 135 140
Pro Ser Ala Thr Gly Gly Gly Gly Gly Asp Thr Pro Thr Ala Thr Gly
145 150 155 160
Gly Gly Gly Ser Gly Gly Gly Gly Thr Pro Thr Ala Thr Gly Gly Gly
                                165 170 175
Ser Gly Gly Thr Pro Thr Ala Thr Gly Gly Gly Glu Gly Gly Val Thr
                        180 185 190
Pro Gln Ile Thr Pro Gln Leu Ala Asn Pro Asn Arg Thr Ser Gly Thr
                195 200 205
Gly Ser Val Ser Asp Thr Ala Gly Ser Thr Glu Gln Ala Gly Lys Ile
        210 215 220
Asn Val Val Lys Asp Thr Ile Lys Val Gly Ala Gly Glu Val Phe Asp
225 230 235 240
Gly His Gly Ala Thr Phe Thr Ala Asp Lys Ser Met Gly Asn Gly Asp
                                245 250 255
Gln Gly Glu Asn Gln Lys Pro Met Phe Glu Leu Ala Glu Gly Ala Thr
                        260 265 270
Leu Lys Asn Val Asn Leu Gly Glu Asn Glu Val Asp Gly Ile His Val
                275 280 285
Lys Ala Lys Asn Ala Gln Glu Val Thr Ile Asp Asn Val His Ala Gln
        290 295 300
Asn Val Gly Glu Asp Leu Ile Thr Val Lys Gly Glu Gly Gly Ala Ala
305 310 315 320
Val Thr Asn Leu Asn Ile Lys Asn Ser Ser Ala Lys Gly Ala Asp Asp
                                325 330 335
Lys Val Val Gln Leu Asn Ala Asn Thr His Leu Lys Ile Asp Asn Phe
                        340 345 350
Lys Ala Asp Asp Phe Gly Thr Met Val Arg Thr Asn Gly Gly Lys Gln
                355 360 365
Phe Asp Asp Met Ser Ile Glu Leu Asn Gly Ile Glu Ala Asn His Gly
        370 375 380
Lys Phe Ala Leu Val Lys Ser Asp Ser Asp Asp Leu Lys Leu Ala Thr
385 390 395 400
Gly Asn Ile Ala Met Thr Asp Val Lys His Ala Tyr Asp Lys Thr Gln
                                405 410 415
Ala Ser Thr Gln His Thr Glu Leu
                        420
```

This protein or polypeptide is about 42.9 kDa.

This hypersensitive response elicitor from Pseudomonas syringae has 1 hypersensitive response eliciting domain. This domain extends, within SEQ. ID. No. 14, from amino acid 45 to amino acid 102, particularly from amino acid 58 to amino acid 92. The acidic unit in the first domain extends, within SEQ. ID. No. 14, from amino acid 45 to amino acid 79, particularly from amino acid 58 to amino acid 79. The alpha-helix in the first domain extends, within SEQ. ID. No. 14, from amino acid 79 to amino acid 102, particularly from amino acid 79 to amino acid 92.

The hypersensitive response elicitor polypeptide or protein derived from Pseudomonas solanacearum has an amino acid sequence corresponding to SEQ. ID. No. 15 as follows:

```
Met Ser Val Gly Asn Ile Gln Ser Pro Ser Asn Leu Pro Gly Leu Gln
1 5 10 15
Asn Leu Asn Leu Asn Thr Asn Thr Asn Ser Gln Gln Ser Gly Gln Ser
                        20 25 30
Val Gln Asp Leu Ile Lys Gln Val Glu Lys Asp Ile Leu Asn Ile Ile
                35 40 45
Ala Ala Leu Val Gln Lys Ala Ala Gln Ser Ala Gly Gly Asn Thr Gly
        50 55 60
Asn Thr Gly Asn Ala Pro Ala Lys Asp Gly Asn Ala Asn Ala Gly Ala
65 70 75 80
Asn Asp Pro Ser Lys Asn Asp Pro Ser Lys Ser Gln Ala Pro Gln Ser
                                85 90 95
Ala Asn Lys Thr Gly Asn Val Asp Asp Ala Asn Asn Gln Asp Pro Met
                        100 105 110
Gln Ala Leu Met Gln Leu Leu Glu Asp Leu Val Lys Leu Leu Lys Ala
                115 120 125
Ala Leu His Met Gln Gln Pro Gly Gly Asn Asp Lys Gly Asn Gly Val
        130 135 140
Gly Gly Ala Asn Gly Ala Lys Gly Ala Gly Gly Gln Gly Gly Leu Ala
145 150 155 160
Glu Ala Leu Gln Glu Ile Glu Gln Ile Leu Ala Gln Leu Gly Gly Gly
                                165 170 175
Gly Ala Gly Ala Gly Gly Ala Gly Gly Gly Val Gly Gly Ala Gly Gly
                        180 185 190
Ala Asp Gly Gly Ser Gly Ala Gly Gly Ala Gly Gly Ala Asn Gly Ala
                195 200 205
Asp Gly Gly Asn Gly Val Asn Gly Asn Gln Ala Asn Gly Pro Gln Asn
        210 215 220
Ala Gly Asp Val Asn Gly Ala Asn Gly Ala Asp Asp Gly Ser Glu Asp
225 230 235 240
Gln Gly Gly Leu Thr Gly Val Leu Gln Lys Leu Met Lys Ile Leu Asn
                                245 250 255
Ala Leu Val Gln Met Met Gln Gln Gly Gly Leu Gly Gly Gly Asn Gln
                        260 265 270
Ala Gln Gly Gly Ser Lys Gly Ala Gly Asn Ala Ser Pro Ala Ser Gly
                275 280 285
Ala Asn Pro Gly Ala Asn Gln Pro Gly Ser Ala Asp Asp Gln Ser Ser
        290 295 300
Gly Gln Asn Asn Leu Gln Ser Gln Ile Met Asp Val Val Lys Glu Val
305 310 315 320
Val Gln Ile Leu Gln Gln Met Leu Ala Ala Gln Asn Gly Gly Ser Gln
                                325 330 335
Gln Ser Thr Ser Thr Gln Pro Met
                        340
```

It is encoded by a DNA molecule having a nucleotide sequence corresponding SEQ. ID. No. 16 as follows:

| | |
|---|---|
| ATGTCAGTCG GAGACATCCA GAGCCCGTCG AACCTCCCGG GTCTGCAGAA CCTGAACCTC | 60 |
| AACACCAACA CCAACAGCCA GCAATCGGGC CAGTCCGTGC AAGACCTGAT CAAGCAGGTC | 120 |
| GAGAAGGACA TCCTCAACAT CATCGCAGCC CTCGTGCAQA AGGCCGCACA GTCGGCGGGC | 180 |
| GGCAACACCG GTAACACCGG CAACGCGCCG GCGAAGGACG GCAATGCCAA CGCGGGCGCC | 240 |
| AACGACCCGA GCAAGAACGA CCCGAGCAAG AGCCAGGCTC CGCAGTCGGC CAACAAGACC | 300 |
| GGCAACGTCG ACGACGCCAA CAACCAGGAT CCGATGCAAG CGCTGATGCA GCTGCTGGAA | 380 |
| GACCTGGTGA AGCTGCTGAA GGCGGCCCTG CACATGCAGC AGCCCGGCGG CAATGACAAG | 420 |
| GGCAACGGCG TGGGCGGTGC CAACGGCGCC AAGGGTGCCG GCGGCCAGGG CGGCCTGGCC | 480 |
| GAAGCGCTGC AQGAGATCGA GCAGATCCTC GCCCAGCTCG GCGGCGGCGG TGCTGGCGCC | 540 |
| GGCGGCGCGG GTGGCGGTGT CGGCGGTGCT GGTGGCGCGG ATGGCGGCTC CGGTGCGGGT | 600 |
| GGCGCAGGCG GTGCGAACGG CGCCGACGGC GGCAATGGCG TGAACGGCAA CCAGGCGAAC | 660 |
| GGCCCGCAGA ACGCAGGCGA TGTCAACGGT CCCAACGGCC CCGATGACGG CAGCGAAGAC | 720 |
| CAGGGCGGCC TCACCGGCGT GCTGCAAAAG CTGATGAAGA TCCTGAACGC GCTGGTGCAG | 780 |
| ATGATGCAGC AAGGCGGCCT CGGCGGCGGC AACCAGGCGC AGGGCCGCTC GAAGGGTGCC | 840 |
| GGCAACGCCT CGCCGGCTTC CGGCGCGAAC CCGGGCGCGA ACCAGCCCGG TTCGGCGGAT | 900 |
| GATCAATCGT CCGGCCAGAA CAATCTGCAA TCCCAGATCA TGGATGTGGT GAAGGAGGTC | 960 |
| GTCCAGATCC TGCAGCAGAT GCTGCCCGCG CAGAACGGCG GCAGCCAGCA GTCCACCTCG | 1020 |
| ACGCAGCCGA TGTAA | 1035 |

Further information regarding the hypersensitive response elicitor polypeptide or protein derived from Pseudomonas solanacearum is set forth in Arlat, M., F. Van Gijsegem, J. C. Huet, J. C. Pemollet, and C. A. Boucher, “PopAl, a Protein which Induces a Hypersensitive-like Response in Specific Petunia Genotypes, is Secreted via the Hrp Pathway of Pseudomonas solanacearum,” EMBO J. 13:543-533 (1994), which is hereby incorporated by reference.

The hypersensitive response elicitor from Pseudomonas solanacearum has 2 hypersensitive response eliciting domains. The first domain extends, within SEQ. ID. No. 15, from amino acid 85 to amino acid 131, particularly from amino acid 95 to amino acid 123. The acidic unit in the first domain extends, within SEQ. BD. No. 15, from amino acid 85 to amino acid 111, particularly from amino acid 95 to amino acid 123. The alpha-helix in the first domain extends, within SEQ. ID. No. 15, from amino acid 85 to amino acid 111, particularly from amino acid 95 to amino acid 111. The second domain extends, within SEQ. ID. No. 15, from amino acid 195 to amino acid 264, particularly from amino acid 229 to amino acid 258. The acidic unit in the second domain extends, within SEQ. ID. No. 15, from amino acid 195 to amino acid 246, particularly from amino acid 229 to amino acid 264. The alpha-helix in the second domain extends, within SEQ. ID. No. 15, from amino acid 246 to amino acid 264, particularly from amino acid 246 to amino acid 258.

The N-terminus of the hypersensitive response elicitor polypeptide or protein from Xanthomonas campestris has an amino acid sequence corresponding to SEQ. ID. No. 17 as follows:

Met Asp Gly Ile Gly Asn His Phe Ser Asn
1 5 10

The hypersensitive response elicitor polypeptide or protein from Xanthomonas campestris pv. pelargonii is heat stable, protease sensitive, and has a molecular weight of 20 kDa. It includes an amino acid sequence corresponding to SEQ. ID. No. 18 as follows:

Ser Ser Gln Gln Ser Pro Ser Ala Gly Ser Glu Gln Gln Leu Asp Gln
1 5 10 15
Leu Leu Ala Met
20

Isolation of Erwinia carotovora hypersensitive response elictor protein or polypeptide is described in Cui et al., “The RsmA Mutants of Erwinia carotovora subsp. carotovora Strain Ecc71 Overexpress hrp N_{Ecc} and Elicit a Hypersensitive Reaction-like Response in Tobacco Leaves,” MPMI, 9(7):565-73 (1996), which is hereby incorporated by reference. The hypersensitive response elicitor protein or polypeptide of Erwinia stewartii is set forth in Ahmad et al., “Harpin is Not Necessary for the Pathogenicity of Erwinia stewartii on Maize,” 8th Int'l. Cong. Molec. Plant-Microbe Interact., July 14-19, 1996 and Ahmad, et al., “Harpin is Not Necessary for the Pathogenicity of Erwinia stewartii on Maize,” Ann. Mtg. Am. Phytopath. Soc., July 27-31, 1996, which are hereby incorporated by reference.

Hypersensitive response elicitor proteins or polypeptides from Phytophthora parasitica, Phytophthora cryptogea, Phytophthora cinnamoni, Phytophthora capsici, Phytophthora megasperma, and Phytophora citrophthora are described in Kaman, et al., “Extracellular Protein Elicitors from Phytophthora: Most Specificity and Induction of Resistance to Bacterial and Fungal Phytopathogens,” Molec. Plant-Microbe Interact., 6(l):15-25 (1993), Ricci et al., “Structure and Activity of Proteins from Pathogenic Fungi Phytophthora Eliciting Necrosis and Acquired Resistance in Tobacco,” Eur. J. Biochem., 183:555-63 (1989), Ricci et al., “Differential Production of Parasiticein, and Elicitor of Necrosis and Resistance in Tobacco, by Isolates of Phytophthora parasitica,” Plant Path. 41:298-307 (1992), Baillreul et al, “A New Elicitor of the Hypersensitive Response in Tobacco: A Fungal Glycoprotein Elicits Cell Death, Expression of Defence Genes, Production of Salicylic Acid, and Induction of Systemic Acquired Resistance,” Plant J., 8(4):551-60 (1995), and Bonnet et al., “Acquired Resistance Triggered by Elicitors in Tobacco and Other Plants,” Eur. J. Plant Path., 102:181-92 (1996), which are hereby incorporated by reference.

Another hypersensitive response elicitor in accordance with the present invention is from Clavibacter michiganensis subsp. sepedonicus which is fully described in U.S. patent application Ser. No. 09/136,625, which is hereby incorporated by reference.

The above elicitors are exemplary. Other elicitors can be identified by growing fungi or bacteria that elicit a hypersensitive response under conditions which genes encoding an elicitor are expressed. Cell-free preparations from culture supernatants can be tested for elicitor activity (i.e. local necrosis) by using them to infiltrate appropriate plant tissues.

Fragments of the above hypersensitive response elicitor polypeptides or proteins as well as fragments of full length elicitors from other pathogens are encompassed by the method of the present invention.

Suitable fragments can be produced by several means. In the first, subclones of the gene encoding a known elicitor protein are produced by conventional molecular genetic manipulation by subcloning gene fragments. The subclones then are expressed in vitro or in vivo in bacterial cells to yield a smaller protein or peptide that can be tested for elicitor activity according to the procedure described below.

As an alternative, fragments of an elicitor protein can be produced by digestion of a full-length elicitor protein with proteolytic enzymes like chymotrypsin or Staphylococcus proteinase A, or trypsin. Different proteolytic enzymes are likely to cleave elicitor proteins at different sites based on the amino acid sequence of the elicitor protein. Some of the fragments that result from proteolysis may be active elicitors of resistance.

In another approach, based on knowledge of the primary structure of the protein, fragments of the elicitor protein gene may be synthesized by using the PCR technique together with specific sets of primers chosen to represent particular portions of the protein. These then would be cloned into an appropriate vector for expression of a truncated peptide or protein.

Chemical synthesis can also be used to make suitable fragments. Such a synthesis is carried out using known amino acid sequences for the elicitor being produced. Alternatively, subjecting a full length elicitor to high temperatures and pressures will produce fragments. These fragments can then be separated by conventional procedures (e.g., chromatography, SDS-PAGE).

An example of suitable fragments of a hypersensitive response elicitor which do elicit a hypersensitive response are Erwinia amylovora fragments including a C-terminal fragment of the amino acid sequence of SEQ. ID. No. 3, an N-terminal fragment of the amino acid sequence of SEQ. ID. No. 3, or an internal fragment of the amino acid sequence of SEQ. ID. No. 3. The C-terminal fragment of the amino acid sequence of SEQ. ID. No. 3 can span amino acids 105 and 403 of SEQ. ID. No. 3. The N-terminal fragment of the amino acid sequence of SEQ. ID. No. 3 can span the following amino acids of SEQ. ID. No. 3: 1 and 98, 1 and 104, 1 and 122, 1 and 168, 1 and 218, 1 and 266, 1 and 342, 1 and 321, and 1 and 372. The internal fragment of the amino acid sequence of SEQ. ID. No. 3 can span the following amino acids of SEQ. ID. No. 3: 76 and 209, 105 and 209, 99 and 209, 137 and 204, 137 and 200, 109 and 204, 109 and 200, 137 and 180, and 105 and 180.

Suitable DNA molecules are those that hybridize to the DNA molecule comprising a nucleotide sequence of SEQ. ID. Nos. 2, 4, 5, 7, 9, 12, 13, and 16 under stringent conditions. An example of suitable high stringency conditions is when hybridization is carried out at 65° C. for 20 hours in a medium containing 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA. Suitable stringency conditions also include hybridization in a hybridization buffer comprising 0.9M sodium citrate (“SSC”) buffer at a temperature of 37° C. where hybridized nucleic acids remain bound when subject to washing the SSC buffer at a temperature of 37° C.; and preferably in a hybridization buffer comprising 20% formamide in 0.9M SSC buffer at a temperature of 42° C. where hybridized nucleic acids remain bound when subject to washing at 42° C. with 0.2× SSC buffer at 42° C.

Variants may be made by, for example, the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

A particularly advantageous aspect of the present invention involves utilizing a protein having a pair or more, particularly 3 or more, coupled domains. These domains can be from different source organisms. When a DNA molecule encoding such a protein is prepared, it can be advantageously used to make transgenic plants. The use of a gene encoding such domains, as opposed to a gene encoding a full length hypersensitive response elicitor, has a number of benefits. Firstly, such a gene is easier to synthesize. More significantly, the use of a plurality of domains together from different source organisms can impart their combined benefits to a transgenic plant.

The DNA molecule encoding the hypersensitive response elicitor polypeptide or protein can be incorporated in cells using conventional recombinant DNA technology. Generally, this involves inserting the DNA molecule into an expression system to which the DNA molecule is heterologous (i.e. not normally present). The heterologous DNA molecule is inserted into the expression system or vector in proper sense orientation and correct reading frame. The vector contains the necessary elements for the transcription and translation of the inserted protein-coding sequences.

U.S. Pat. No. 4,237,224 to Cohen and Boyer, which is hereby incorporated by reference, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture.

Recombinant genes may also be introduced into viruses, such as vaccina virus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC 1084, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif, which is hereby incorporated by reference), pQE, pIH821, pGEX, pET series (see F.W. Studier et. al., “Use of T7 RNA Polymerase to Direct Expression of Cloned Genes,” Gene Expression Technology vol. 185 (1990), which is hereby incorporated by reference), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, New York (1989), which is hereby incorporated by reference.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promotor which is a DNA sequence that directs the binding of RNA polymerase and thereby promotes mRNA synthesis. The DNA sequences of eucaryotic promotors differ from those of procaryotic promotors. Furthermore, eucaryotic promotors and accompanying genetic signals may not be recognized in or may not function in a procaryotic system, and, further, procaryotic promotors are not recognized and do not function in eucaryotic cells.

Similarly, translation of MRNA in procaryotes depends upon the presence of the proper procaryotic signals which differ from those of eucaryotes. Efficient translation of MRNA in procaryotes requires a ribosome binding site called the Shine-Dalgamo (“SD”) sequence on the MIRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference.

Promotors vary in their “strength” (i.e. their ability to promote transcription). For the purposes of expressing a cloned gene, it is desirable to use strong promotors in order to obtain a high level of transcription and, hence, expression of the gene. Depending upon the host cell system utilized, any one of a number of suitable promotors may be used. For instance, when cloning in E. coli, its bacteriophages, or plasmids, promotors such as the T7 phage promoter, lac promotor, trp promotor, recA promotor, ribosomal RNA promotor, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promotor or other E. coli promotors produced by recombinant DNA or other synthetic DNA techniques may be used to provide for transcription of the inserted gene.

Bacterial host cell strains and expression vectors may be chosen which inhibit the action of the promotor unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in procaryotic cells. These transcription and translation initiation signals may vary in “strength” as measured by the quantity of gene specific messenger RNA and protein synthesized, respectively. The DNA expression vector, which contains a promotor, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Once the isolated DNA molecule encoding the hypersensitive response elicitor polypeptide or protein has been cloned into an expression system, it is ready to be incorporated into a host cell. Such incorporation can be carried out by the various forms of transformation noted above, depending upon the vector/host cell system. Suitable host cells include, but are not limited to, plant cells as well as prokaryotic and eukaryotic cells, such as bacteria, virus, yeast, mammalian, insect cells, and the like.

The present invention further relates to methods of imparting disease resistance to plants, enhancing plant growth, effecting insect control and/or imparting stress resistance to plants. These methods involve applying a hypersensitive response elicitor polypeptide or protein to all or part of a plant or a plant seed under conditions where the polypeptide or protein contacts all or part of the cells of the plant or plant seed. Alternatively, the hypersensitive response elicitor protein or polypeptide can be applied to plants such that seeds recovered from such plants themselves are able to impart disease resistance in plants, to enhance plant growth, to effect insect control, and/or to impart stress resistance.

As an alternative to applying a hypersensitive response elicitor polypeptide or protein to plants or plant seeds in order to impart disease resistance in plants, to effect plant growth, to control insects, and/or to impart stress resistance to the plants or plants grown from the seeds, transgenic plants or plant seeds can be utilized. When utilizing transgenic plants, this involves providing a transgenic plant transformed with a DNA molecule encoding a hypersensitive response elicitor polypeptide or protein and growing the plant under conditions effective to permit that DNA molecule to impart disease resistance to plants, to enhance plant growth, to control insects, and/or to impart stress resistance. Alternatively, a transgenic plant seed transformed with a DNA molecule encoding a hypersensitive response elicitor polypeptide or protein can be provided and planted in soil. A plant is then propagated from the planted seed under conditions effective to permit that DNA molecule to impart disease resistance to plants, to enhance plant growth, to control insects, and/or to impart stress resistance.

The method of the present invention can be utilized to treat a wide variety of plants or their seeds to impart disease resistance, enhance growth, control insects, and/or to impart stress resistance. Suitable plants include dicots and monocots. More particularly, useful crop plants can include: alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. Examples of suitable ornamental plants are: Arabidopsis thaliana, Saintpaulia, petunia, pelargonium, poinsettia, chrysanthemum, carnation, and zinnia.

With regard to the use of the hypersensitive response elicitor protein or polypeptide of the present invention in imparting disease resistance, absolute immunity against infection may not be conferred, but the severity of the disease is reduced and symptom development is delayed. Lesion number, lesion size, and extent of sporulation of fungal pathogens are all decreased. This method of imparting disease resistance has the potential for treating previously untreatable diseases, treating diseases systemically which might not be treated separately due to cost, and avoiding the use of infectious agents or environmentally harmful materials.

The method of imparting pathogen resistance to plants in accordance with the present invention is useful in imparting resistance to a wide variety of pathogens including viruses, bacteria, and fungi. Resistance, inter alia, to the following viruses can be achieved by the method of the present invention: Tobacco mosaic virus and Tomato mosaic virus. Resistance, inter alia, to the following bacteria can also be imparted to plants in accordance with present invention: Pseudomonas solancearum, Pseudomonas syringae pv. tabaci, and Xanthamonas campestris pv. pelargonii. Plants can be made resistant, inter alia, to the following fungi by use of the method of the present invention: Fusarium oxysporum and Phytophthora infestans.

With regard to the use of the hypersensitive response elicitor protein or polypeptide of the present invention to enhance plant growth, various forms of plant growth enhancement or promotion can be achieved. This can occur as early as when plant growth begins from seeds or later in the life of a plant. For example, plant growth according to the present invention encompasses greater yield, increased quantity of seeds produced, increased percentage of seeds germinated, increased plant size, greater biomass, more and bigger fruit, earlier fruit coloration, and earlier fruit and plant maturation. As a result, the present invention provides significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land.

Another aspect of the present invention is directed to effecting any form of insect control for plants. For example, insect control according to the present invention encompasses preventing insects from contacting plants to which the hypersensitive response elicitor has been applied, preventing direct insect damage to plants by feeding injury, causing insects to depart from such plants, killing insects proximate to such plants, interfering with insect larval feeding on such plants, preventing insects from colonizing host plants, preventing colonizing insects from releasing phytotoxins, etc. The present invention also prevents subsequent disease damage to plants resulting from insect infection.

The present invention is effective against a wide variety of insects. European corn borer is a major pest of corn (dent and sweet corn) but also feeds on over 200 plant species including green, wax, and lima beans and edible soybeans, peppers, potato, and tomato plus many weed species. Additional insect larval feeding pests which damage a wide variety of vegetable crops include the following: beet armnyworm, cabbage looper, corn ear worm, fall armyworm, diamondback moth, cabbage root maggot, onion maggot, seed corn maggot, pickleworm (melonworm), pepper maggot, and tomato pinworm. Collectively, this group of insect pests represents the most economically important group of pests for vegetable production worldwide.

Another aspect of the present invention is directed to imparting stress resistance to plants. Stress encompasses any environmental factor having an adverse effect on plant physiology and development. Examples of such enviromnental stress include climate-related stress (e.g., drought, water, frost, cold temperature, high temperature, excessive light, and insufficient light), air polllution stress (e.g., carbon dioxide, carbon monoxide, sulfur dioxide, NOₓ, hydrocarbons, ozone, ultraviolet radiation, acidic rain), chemical (e.g., insecticides, fungicides, herbicides, heavy metals), and nutritional stress (e.g., fertilizer, micronutrients, macronutrients). Use of hypersensitive response elicitors in accordance with the present invention impart resistance to plants against such forms of environmental stress.

The method of the present invention involving application of the hypersensitive response elicitor polypeptide or protein can be carried out through a variety of procedures when all or part of the plant is treated, including leaves, stems, roots, propagules (e.g., cuttings), etc. This may (but need not) involve infiltration of the hypersensitive response elicitor polypeptide or protein into the plant. Suitable application methods include high or low pressure spraying, injection, and leaf abrasion proximate to when elicitor application takes place. When treating plant seeds, in accordance with the application embodiment of the present invention, the hypersensitive response elicitor protein or polypeptide can be applied by low or high pressure spraying, coating, immersion, or injection. Other suitable application procedures can be envisioned by those skilled in the art provided they are able to effect contact of the hypersensitive response elicitor polypeptide or protein with cells of the plant or plant seed. Once treated with the hypersensitive response elicitor of the present invention, the seeds can be planted in natural or artificial soil and cultivated using conventional procedures to produce plants. After plants have been propagated from seeds treated in accordance with the present invention, the plants may be treated with one or more applications of the hypersensitive response elicitor protein or polypeptide to impart disease resistance to plants, to enhance plant growth, to control insects on the plants, and/or impart stress resistance.

The hypersensitive response elicitor polypeptide or protein can be applied to plants or plant seeds in accordance with the present invention alone or in a mixture with other materials. Alternatively, the hypersensitive response elicitor polypeptide or protein can be applied separately to plants with other materials being applied at different times.

A composition suitable for treating plants or plant seeds in accordance with the application embodiment of the present invention contains a hypersensitive response elicitor polypeptide or protein in a carrier. Suitable carriers include water, aqueous solutions, slurries, or dry powders. In this embodiment, the composition contains greater than 500 nM hypersensitive response elicitor polypeptide or protein.

Although not required, this composition may contain additional additives including fertilizer, insecticide, fungicide, nematacide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and abrading agents. These materials can be used to facilitate the process of the present invention. In addition, the hypersensitive response elicitor polypeptide or protein can be applied to plant seeds with other conventional seed formulation and treatment materials, including clays and polysaccharides.

In the alternative embodiment of the present invention involving the use of transgenic plants and transgenic seeds, a hypersensitive response elicitor polypeptide or protein need not be applied topically to the plants or seeds. Instead, transgenic plants transformed with a DNA molecule encoding a hypersensitive response elicitor polypeptide or protein are produced according to procedures well known in the art.

The vector described above can be microinjected directly into plant cells by use of micropipettes to transfer mechanically the recombinant DNA. Crossway, Mol. Gen. Genetics, 202:179-85 (1985), which is hereby incorporated by reference. The genetic material may also be transferred into the plant cell using polyethylene glycol. Krens, et al., Nature, 296:72-74 (1982), which is hereby incorporated by reference.

Another approach to transforming plant cells with a gene which imparts resistance to pathogens is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford et al., which are hereby incorporated by reference. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells.

Yet another method of introduction is fusion of protoplasts with other entities, either minicells, cells, lysosomes or other fusible lipid-surfaced bodies. Fraley, et al., Proc. Natl. Acad. Sci. USA, 79:1859-63 (1982), which is hereby incorporated by reference.

The DNA molecule may also be introduced into the plant cells by electroporation. Fromm et al., Proc. Natl. Acad. Sci. USA, 82:5824 (1985), which is hereby incorporated by reference. In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate.

Another method of introducing the DNA molecule into plant cells is to infect a plant cell with Agrobacterium tumefaciens or A. rhizogenes previously transformed with the gene. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C.

Agrobacterium is a representative genus of the gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue.

Heterologous genetic sequences can be introduced into appropriate plant cells, by means of the Ti plasmid of A. tumefaciens or the Ri plasmid of A. rhizogenes. The Ti or Ri plasmid is transmitted to plant cells on infection by Agrobacterium and is stably integrated into the plant genome. J. Schell, Science, 237:1176-83 (1987), which is hereby incorporated by reference.

After transformation, the transformed plant cells must be regenerated.

Plant regeneration from cultured protoplasts is described in Evans et al., Handbook of Plant Cell Cultures, Vol. 1: (MacMillan Publishing Co., New York, 1983); and Vasil I.R. (ed.), Cell Culture and Somatic Cell Genetics of Plants, Acad. Press, Orlando, Vol. 1, 1984, and Vol. III (1986), which are hereby incorporated by reference.

It is known that practically all plants can be regenerated from cultured cells or tissues, including but not limited to, all major species of sugarcane, sugar beets, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

After the expression cassette is stably incorporated in transgenic plants, it can be transferred to other plants by sexual crossing. Any of a number of standard breeding techniques can be used, depending upon the species to be crossed.

Once transgenic plants of this type are produced, the plants themselves can be cultivated in accordance with conventional procedure with the presence of the gene encoding the hypersensitive response elicitor resulting in disease resistance, enhanced plant growth, control of insects on the plant, and/or stress resistance. Alternatively, transgenic seeds are recovered from the transgenic plants. These seeds can then be planted in the soil and cultivated using conventional procedures to produce transgenic plants. The transgenic plants are propagated from the planted transgenic seeds under conditions effective to impart disease resistance to plants, to enhance plant growth, to control insects, and/or to impart stress resistance. While not wishing to be bound by theory, such disease resistance, growth enhancement, insect control, and/or stress resistance may be RNA mediated or may result from expression of the elicitor polypeptide or protein.

When transgenic plants and plant seeds are used in accordance with the present invention, they additionally can be treated with the same materials as are used to treat the plants and seeds to which a hypersensitive response elicitor polypeptide or protein is applied. These other materials, including hypersensitive response elicitors, can be applied to the transgenic plants and plant seeds by the above-noted procedures, including high or low pressure spraying, injection, coating, and immersion. Similarly, after plants have been propagated from the transgenic plant seeds, the plants may be treated with one or more applications of the hypersensitive response elicitor to impart disease resistance, enhance growth, control insects, and/or to impart stress resistance. Such plants may also be treated with conventional plant treatment agents (e.g., insecticides, fertilizers, etc.).

### EXAMPLES

Example 1—Bacterial Strains and Plasmids

Escherichia coli DH5 and BL2 1 were purchased from Gibco BRL (Rockville, Md.) and Novagen (Madison, Wis.) respectively.

pET28 plasmids were from Novagen (Madison, Wis.).

All restriction enzymes (e.g., NdeI and HindlIl), T4 DNA ligase, Calf intestinal alkaline phosphatase (CIP), and PCR reagents were from Gibco BRL (Rockville, Md.).

Oligonucleotides were synthesized by Lofstrand Labs Ltd (Gaithersburg, Md.).

Chemically synthesized polypeptides were synthesized by Bio-Synthesis (Lewisville, Tex.).

Example 2—Construction of Truncated Gene Encoding Harpin

Fragments of genes encoding harpin proteins were constructed in pET28 vector and expressed in E. Coli as follows;

1. HrpN fragments were PCR amplified from the pCPP2139 plasmid (Cornell University, Ithaca, N.Y.) and cloned into pET28 vector.

2. HrpZ fragments were PCR amplified from the pSYH10 plasmid (Cornell University, Ithaca, NY) and cloned into pET28 vector.

3. PopA fragments were PCR amplified from the pBS::popA plasmid (Cornell University, Ithaca, N.Y.) and cloned into pET28 vector.

4. HrpW fragments were PCR amplified from the pCPP1233 plasmid (Cornell University, Ithaca, N.Y.) and cloned into pET28 vector.

All truncated fragments were amplified by PCR with full length harpin DNA as the template.

Oligonucleotides corresponding to the truncated N-terminal sequence were started /modified with a Nde I site (which serves as an initiation codon of methionine (ATG)). Oligonucleotides corresponding to a C-terminal sequence contained a UAA stop codon followed by a Hind III site.

PCR was carried in a 0.5 ml tube with GeneAmpTM 9600 and 9700 (PE Applied Biosystems, Branchburg, N.J.). 45 IIl of SuperMix™ (Gibco BRL, Rockville, Md.) was mixed with 20 pmoles of each pair of DNA primers, 10 ng of full length harpin DNA, and diH₂O to fill the final volume to 50 μl. After heating the mixture at 95° C. for 2 min., PCR was performed for 30 cycles at 94° C. for 1 min., 58° C. for 1 min. and 72° C. for 1.5 min. Amplified DNAs were purified with QlAquick PCR purification kit (QIAGEN Inc., Vlencia, Calif.), digested with Nde I and Hind III at 37° C. for 5 hours, extracted once with phenol:chloroform:isoamylalcohol (25:24:1), and precipitated with ethanol. 5 μg of pET28(b) vector DNA was digested with 15 units of Nde I and 20 units of Hind III at 37° C. for 3 hours followed with calf intestinal alkaline phosphatase treatment for 30 min. at 37° C. to reduce the background resulting from incomplete single enzyme digestion. Digested vector DNA was purified with the QlAquick PCR purification kit and directly used for ligation. Ligation was carried at 14° C. for 12 hours in a 15 Vtl mixture containing about 50 to 100 ng of digested pET28(b), 10 to 30 ng of targeted PCR fragments, and 1 unit of T4 DNA ligase. 5 μl of ligation solution was added to 100 μl of DH5α/XL1-Blue competent cells, placed in 15 ml Falcon tube, and incubated on ice for 30 min. After heat shock at 42° C. for 45 seconds, 0.9 ml SOC solution (20 g bacto-tryptone, 5 g bacto-yeast extracts, 0.5 g NaCl, 20 mM glucose in one liter) was added into the tube and incubated at 37° C. for 1 hour. 20 μl of transformed cells were plated onto LB agar plate with 30 μg/ml of kanamycin and incubated at 37° C. for 14 hours. Single colonies were transferred to 3 ml LB-media and incubated overnight at 37° C. Plasmid DNA was prepared in a 2 ml culture with QIAprep Miniprep kit according to the manufacture's instruction. The DNA sequence of truncated harpin constructions was verified with restriction enzyme analysis and sequencing analysis. Plasmids with the desired DNA sequence were transferred into the BL21 strain with a standard chemical transformation method as indicated above.

Example 3—Expression of Proteins

A single clone of E. coli with a constructed gene was grown overnight at 37° C. in LB with kanamycin. A proper amount of overnight culture was transferred to 50 to 500 ml LB and incubated at 37° C. until OD600 reached 0.5 to 0.8. ITPG was added to the culture which was further incubated at room temperature for a period of 5 hour to overnight. Alternatively, a proper amount of overnight culture was transferred to 50 to 500 ml of ½ TB with lactose medium (6 g bacto-trypton, 12 g bacto-yeast extract, 75 g lactose in one liter). After incubation at 37° C. until the OD600 reached 0.5 to 0.8, the culture was incubated at room temperature for a period of 5 hours to overnight.

All bacterial cells were harvested by centrifugation and resuspended in 1:5 TE buffer (10 mM Tris, pH 8.5 and 1 mM EDTA). The cells were disrupted by sonication and clarified by centrifugation. Supernatants were then infiltrated into tobacco leaves for HR testing.

Heat treatment (i.e. boiling for 1 to 10 min.) was used to achieve further purification.

All truncated fragments of genes encoding harpin protein were expressed in E. colil BL-2 1, DE3 strain with an N-terminal His-tag and 20 to 21 amino acid residues generated from the expression vector sequence. The His-tag sequence did not affect the HR activity of the proteins. In some cases, Ni-Agarose beads were added into supernatant solution and mixed at 4° C. to room temperature for a period of 30 min. to overnight. The proteins bound to the Ni-Agarose beads were washed by 0.1 M imidazole buffer, and proteins were eluted with 0.6 to 1.0 M imidazole. After dialysis against 10 mM Tris, pH 8.5 buffer, the proteins were infiltrated into tobacco leaves for HR testing.

For proteins expressed in E. coli that were difficult to dissolve in water, total cells were resuspended and sonicated in 8 M urea buffer (0. IM Na-phosphate, 10 mM Tris buffer, pH8.0). The total cell lysate was centrifuged, and supernatants were collected. Ni-agarose was added into the supernatants and mixed gently at room temperature for 30 min. The Ni-agarose resin was washed with buffer (8 M urea, 0.1 M Na-phosphate, 10 mM Tris buffer, pH6.3). The proteins were eluted with elution buffer (8 M urea, 0.1 M EDTA, 0.1 M Na-phosphate, 10 mM Tris buffer, pH 6.3) and dialyzed against buffer (pH 8.5, 10 mM Tris) with stepwise decreased urea. If the proteins still were insoluble in buffer, the solution pH was adjusted to 9 to 11 and sonicated at room temperature for 1 to 5 min.

Chemically synthesized polypeptides were dissolved in 10 mM Tris, pH 6.5 to 11 buffers depending on their solubility.

A hypersensitive response (“HR”) assay was performed by infiltration of 0.1 to 0.3 ml of serial diluted protein solutions into tobacco leaves (cv. Xanth). All HR data shown in these examples were recorded from 48 hours after infiltration.

Example 4—Quantification of Proteins

All expressed proteins were checked with pre-cast 4-20% SDS polyacrylamide gel electrophoresis (SDS-PAGE) from Novex (San Diego, Calif.). After electrophoresis, the gel was stained with Coomasssie R-250 solution (0.1% Coomassie R-250, 10% Acetate Acid, 40% ethanol) for 1 to 4 hours and distained with distaining solution (8% acetate acid and 25% ethanol) overnight. The density of corresponding bands were compared to standard proteins, which were either purchased from Novex or were from quantitative standard harpin protein produced by Eden Bioscience (Bothell, Wash.).

Example 5—Classification of Harpin Proteins

Since harpin proteins share common biochemical and biophysical characteristics as well as biological functions, based on their unique properties, HR elicitors from various pathogenic bacteria should be viewed as belonging to a new protein family-i.e. the harpin protein family. The harpin protein can be classified into at least four subfamilies based on their primary structure and isolated sources. As set forth in Table 1, those subfamilies are identified by the designation N, W, Z, A, etc.

TABLE 1
Subfamilies of Harpin Proteins

| | | | | | | Core |
|---|---|---|---|---|---|---|
| Harpin | | Classified | | Amino | Heat | struc- |
| proteins | Isolated Source | Subfamily | pI | acids | stable | ture |
| HrpN_{Ea} | E. amylovora | N | 4.42 | 403 | Yes | No |
| HrpN_{Ech} | E. chrysanthemi | N | 6.51 | 340 | Yes | No |
| HrpN_{Ecc} | E. carotovora | N | 5.82 | 356 | Yes | No |
| HrpN_{Est} | E. stewartii | N | N/A | N/A | Yes | No |
| HrpW_{Pss} | P. syringae | W | 4.43 | 424 | Yes | No |
| HrpW_{Ea} | E. amylovora | W | 4.46 | 447 | Yes | No |
| HrpZ_{Pss} | P. syringae | Z | 3.95 | 341 | Yes | No |
| PopA1 | R. solanacearum | A | 4.16 | 344 | Yes | No |

Example 6—Analysis of the Structural Units of an HR Domain

The sequence of amino acids that alone could elicit a hypersensitive response in plants (i.e. HR domains) has been investigated in different ways. It was reported that a carboxyl-terminal 148 amino acid portion of HrpZ_{Pss} is sufficient and necessary for HR (He et al., “Pseudomonas Syringae pv. Syringae Harpinₚₛₛ: A Protein that is Secreted via the Hrp Pathway and Elicits the Hypersensitive Response in Plants,” Cell 73:1255-1266.(1993), which is hereby incorporated by reference). With truncated HrpZ fragments, it was determined that an N-terminal 109 amino acids and C-terminal 216 amino acids of HrpZ_{Pss}, respectively, were found to elicit HR (Alfano et al., “Analysis of the Role of the Pseudomonas Syringae pv. Syringae HrpZ Harpin in Elicitation of the Hypersensitive Response in Tobacco Using Functionally Non-polar hrpZ Deletion Mutations, Truncated HrpZ Fragments, and himA Mutations,” Molecular Microbiology 19:715-728 (1996), which is hereby incorporated by reference). Jin et al., “A Truncated Fragment of Harpinp,, Induces Systemic Resistance to Xanthomonas campestris pv. Oryzae in Rice,” Physiological and Molecular Plant Pathology 51:243-257 (1997), which is hereby incorporated by reference, reported that a truncated HrpZ_{Pss}, with an N-terminal of 137 amino acids elicited a hypersensitive response in tobacco and induced systemic acquired resistance (i.e. SAR) in rice. After digestion with protease, a hypersensitive response active fragment of HrpNEa was isolated and found to span amino acids 137 to 204 of HrpNEa. It was found that a 98 residue of N-terminal HrpNEa fragment was the smallest bacterially produced peptide that displayed HR-eliciting activity (Laby, “Molecular Studies on Interactions Between Erwinia Amylovora and its Host and Non-host Plants,” Doctoral Thesis in Cornell University (1997), which is hereby incorporated by reference).

A series of HrpN_{Ea} fragments have been generated with His-tag fusion at the N-terminal of the polypeptides and a polypeptide (HrpN_{Ea}37180), located at position of 137 to 180 amino acid residue of HrpN_{Ea}, was identified to elicit HR activity in tobacco.

Example 7—Analysis of Secondary Structure of HR Domains

The DNA and primary protein sequence of the HrpN_{Ea}137180 show no any homologues among other hypersensitive response elicitors.

Analyses of the secondary structure of the fragment of HrpN_{Ea}37180 revealed, with the aid of the computer program Clone Manger5 (Scientific & Educational Software, Durham, N.C.), that there was a beta-form, a beta-turn, and unordered forms. One typical α-helical segment of residues at 157-170 was found in the HrpN_{Ea}37180 polypeptide. To determine the function of this structure, polypeptides with a disrupted α-helical structure were generated and hypersensitive response results were evaluated. As shown in Table 2, a complete alpha-helix unit (H unit), probably with a length greater than 12 amino acid residues, is need for hypersensitive response activity.

TABLE 2
Effect of Alpha-helix Structure

| Fragment name | Amino acid | HR* | Structure | Source |
|---|---|---|---|---|
| HrpN_{Ea}137180 | 137-180 (44) | + | Complete H | E.coli |
| | pI = 3.10 | <5 μg/ml | | expressed |
| | | | | peptide |
| HrpN_{Ea}137166 | 137-166 (30) | − | disrupted H | Synthesized |
| | pI = 3.29 | | | peptide |
| HrpN_{Ea}76168 | 76-168 | − | disrrpted H | E.coli |
| | pI = 3.39 | | | expressed |
| | | | | peptide |

The α-helical unit plays an important role in hypersensitive response activity; however, it was found that an a-helix unit alone did not achieve HR (Table 3).

Therefore, hypersensitive response eliciting domains contain more than one structure unit. Besides the core a-helical unit, there is an acidic unit that has no typical secondary structure feature but is rich in acidic amino acids. This relaxed structure, having a sheet and random turn, is designated as an acidic unit (A unit).

Although the acidic unit is important in achieving a hypersensitive response, it alone, like the a-helical unit alone, did not elicit a hypersensitive response.

A synthetic polypeptide, HrpN_{Ea}140176, that included both A and H structure, spanning amino acids 140 to 176 of HrpN_{Ea}, gave full activity of HR. Sequence analysis by major search engines revealed no global primary sequence similarity in the databases to HrpN_{Ea}140176, even among the harpin protein families.

TABLE 3
Effect of Acidic Unit on Hypersensitive Response (HR) Activity

| | | | Structure | |
|---|---|---|---|---|
| Fragment name | Amino acid | HR* | (A or H)** | Source |
| HrpN_{Ea}140176 | 140-176 (37) | + | A + H | Synthesized |
| | pI = 3.17 | <5 μg/ml | | peptide |
| HrpN_{Ea}157170 | 157-170 (14) | − | H | Synthesized |
| | pI = 6.94 | | peptide | |
| HrpN_{Ea}137156 | 137-156 (20) | − | A | Synthesized |
| | pI = 2.67 | | | peptide |

Example 8—Hypersensitive Response Domain Structure of HrpNEa

Four a-helical regions with at least 12 amino acid residues were found in HrpN_{Ea} based on computer analysis with the program Clone Manager 5 (Scientific & Educational Software, Durham, N.C.), which predicts the secondary structure of protein from the primary sequence by the method of Garnier-Osguthorpe-Robson.

It is believed that a hypersensitive response domain includes two structural units, the ax-helix (H) and the acidic unit (A). Another hypersensitive response domain, spanning amino acids 43 to 70 in HrpNEa, was found. A minimal sequence of 12 to 14 AA residues of both the H and A units is believed to be needed. The chemically synthesized polypeptide of HrpN_{Ea}4370 gave full HR activity in tobacco. Thus, a second HR domain has been discovered based on purely secondary structure analysis and prediction.

To further test the hypothesis that the A and H units are needed to achieve a hypersensitive response, an approach of unit exchange (i.e. swapping an acidic unit from one HR domain to another HR domain) was designed. A polypeptide of HrpN_{Ea}Dswap, which consisted of the acidic unit of a hypersensitive response domain (HrpN_{Ea}140176), spanning amino acids 136 to 156 of HrpN_{Ea}, and the α-helical unit of another hypersensitive response domain (HrpN_{Ea}4370), spanning amino acids 57 to 70 of HrpN_{Ea}, was chemically synthesized. This polypeptide swapped two structural units of A and H between two hypersensitive response domains of HrpN_{Ea}4370 and HrpN_{Ea}140176. The HrpN_{Ea}Dswap gave a hypersensitive response activity in tobacco (Table 4). This result shows that the structural characteristic of an HR domain determines its activity, and structural analysis can be used to determine hypersensitive response activity.

TABLE 4
Two Structural Units Determine Hypersensitive Response Activity

| Fragment | | | | |
|---|---|---|---|---|
| name | Amino acid | HR | Structure Type | Source |
| HrpN_{Ea}4370 | 43-70 (28) | + | A + H | Synthesized |
| | pI = 3.09 | <5 μg/ml | | peptide |
| | | | | Partial |
| | | | | soluble |
| HrpN_{Ea}D_{swap} | HrpN136156 | <20 μg/ml | A unit from | Synthesized |
| | (A)+ | | HrpN_{Ea}140176 + | peptide |
| | HrpN5770 | | H unit from | Partial |
| | (H) | | HrpN_{Ea}4370 | soluble |
| | pI = 2.67 | | | |

Example 9—Prediction of Hypersensitive Response Domains Among Proteins in Harpin Family

The secondary structure which indicates the presence of a hypersensitive response domain in HrpNEa was used to identify other harpin proteins, including proteins classified as different subfamilies. Structural prediction of a hypersensitive response domain among harpin proteins was carried according to following criteria:

1. There are two structural units in a hypersensitive response domain, including:

a. A stable oc-helix unit with 12 or more amino acids in length and

b. An hydrophilic, acidic unit with 12 or more amino acids in length which could be a beta-form, a beta-turn, and unordered forms.

2. The pI of a hypersensitive response domain should be acidic and, in general, below 5.

3. The minimal size of an HR domain is from about 28 to 40 AA residues.

Putative HR domains have been identified to fit the criteria by computer analysis among harpin protein family (Table 5).

TABLE 5
Predication of Hypersensitive Response Domains Among Harpin
Proteins

| HR domain | Isolated Source | Predicted region* | pI | Structure |
|---|---|---|---|---|
| HrpN_{Ea}-1 | E. amylovora | 43-70 | 3.09 | A + H |
| HrpN_{Ea}-2 | E. amylovora | 140-176 | 3.17 | A + H |
| HrpN_{Ech}-1 | E. chrysanthemi | 78-118 | 5.25 | A + H |
| HrpN_{Ech}-2 | E. chrysanthemi | 256-295 | 4.62 | A + H |
| HrpN_{Ecc}-1 | E. carotovora | 25-63 | 4.06 | A + H |
| HrpN_{Ecc}-2 | E. carotovora | 101-140 | 3.00 | A + H |
| HrpW_{Pss}-1 | P. syringae | 52-96 | 4.32 | A + H |
| HrpW_{Ea}-1 | E. amylovora | 10-59 | 4.53 | A + H |
| HrpZ_{Pss}-1 | P. syringae | 97-132 | 3.68 | A + H |
| HrpZ_{Pss}-2 | P. syringae | 153-189 | 3.67 | A + H |
| HrpZ_{Pss}-3 | P. syringae | 271-308 | 3.95 | A + H |
| PopA1_{Rs}-1 | R.solanacearum | 92-125 | 3.75 | A + H |
| PopA1_{Rs}-2 | R.solanacearum | 206-260 | 3.62 | A + H |

*Amino acid residue position

Example 10—Hypersensitive Response Activity of Select Synthesized Polypeptides

Polypeptides were produced by expression in either E. coli or by chemical synthesis. Based on prediction of solubility and stability of a particular peptide, in some cases, a broader region of AA residues in addition to the essential units were also synthesized to increase solubility of the peptides. The identification of HR domains among four subfamilies of harpin protein demonstrated this (Table 6).

TABLE 6
Hypersensitive Response Activity of Select Synthesized Polypeptides

| HR | | Synthesized | | | HR |
|---|---|---|---|---|---|
| domain | Isolated Source | region | pI | Source | activity |
| HrpN_{Ea}-1 | E. amylovora | 43-70 | 3.09 | Chemical | + < 5 |
| | | | | Synthesized | μg/ml |
| HrpN_{Ea}-2 | E. amylovora | 140-176 | 3.17 | Chemical | + < 5 |
| | | | | Synthesized | μg/ml |
| HrpW_{Ea}-2 | E. amylovora | 10-59 | 4.53 | E.coli | + < 5 |
| | | | | expressed | μg/ml |
| HrpZ_{Pss}-1 | P. syringae | 97-132 | 3.68 | Chemical | + < 20 |
| | | | | Synthesized | μg/ml |
| HrpZ_{Pss}-1 | P. syringae | 153-189 | 3.69 | E.coli | + < 5 |
| | | | | expressed | μg/ml |
| PopA1_{Rs}-1 | R. | 92-125 | 3.75 | Chemical | + < 5 |
| | solanacearum | | | Synthesized | μg/ml |
| PopA1_{Rs}-2 | R. | 206-260 | 3.62 | E.coli | + < 5 |
| | solanacearum | | | expressed | μg/ml |

Example 11—Construction of Hypersensitive Response Domains in a Protein Expression Cassette

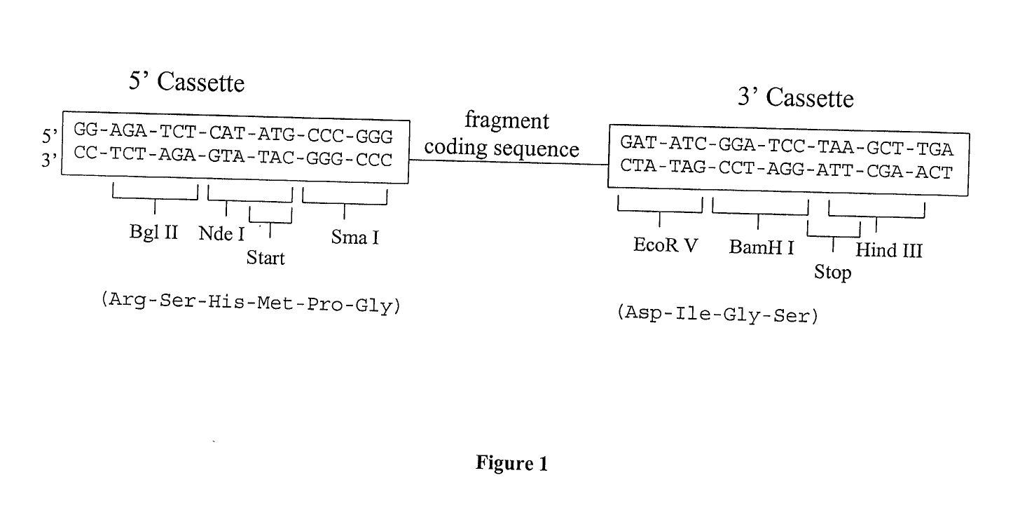

Polypeptides with a harpin protein hypersensitive response domain were expressed in E. coli. PCR was used to amplify desired areas of genes encoding harpin proteins and cloned into an expression vector, e.g. pET28a. A pair of PCR primers with unique flanking sequences were designed to create a universal expression cassette, as shown in FIG. 1, for expression of a fragment of harpin protein. Each amplified DNA fragment has a protein translation start codon of ATG in a restriction enzyme Nde I site which might add an extra amino acid of methionine into a polypeptide. Each amplified DNA fragment has a protein translation stop codon of TAA. Each amplified fragment contained two restriction enzyme sites of EcoR V and Sma I, which gave 4 extra in-frame amino acids expressed as Pro-Gly at the N-terminal and Asp-Ile at the C-terminal, respectively. Those two sites are essential to allow two or more expression cassettes to be linked in a specific order and in frame with a minimum number of amino acids being introduced. Cassette A was first digested by EcoR V, ligated to cassette B, and digested with Sma I to produce a new expression cassette C which coupled the two fragments together with two extra amino acids (i.e. Asp-Gly), which are common amino acids in hypersensitive response domains. The newly formed cassette C still contained the same 5′ and 3′ flanking sequences as original cassettes A and B and maintained the ability to be coupled by another cassette. Bg1 II and Bam HI sites in the cassette permit the cassette to be linked in frame into a cancatomer with a correct orientation. The strategy is that digestion of DNA with Bg1 II and Bam HI results in compatible ends that would be ligated with each other but could not be cut by either enzymes after ligation. For example, a DNA fragment encoding a hypersensitive response domain in a cassette could be digested by restrictions enzymes of Bg1 II and Bam Hi separately, digested DNA fragments could be ligated in a ligation solution also including both Bg1 II and Bam HI enzymes, any ligated ends with Bg1 II or Bam HI sites could be digested by the enzymes, and only those ligated sites between Bg1 II and Bam HI could remain.

Example 12—Building Blocks for Creating Superharpins that have Higher Biological Efficacy

Hypersensitive response domains were identified and isolated from several harpin proteins. With the combination of those HR domains, new polypeptides (i.e. superharpins) that have higher HR potency and have enhanced ability to induce disease resistance, impart insect resistance, enhance growth, and achieve environmental stress tolerance. Superharpins could be one HR domain repeat units (cancatomer), different combinations of HR domains, and/or biologically active domains from other elicitors. Part of the domains from different harpin proteins and other elicitors were constructed into the universal expression cassette as shown on Example 11 and designated as superharpin building blocks. Table 7 lists some superharpin building blocks which were expressed in pET-28a(+) vector with a His-tag sequence at their N-terminal.

TABLE 7
Superharpin Building Blocks including pET-28a(+) his-tag Leader
Sequence

| Domain | | MW | | | | (Structurally) |
|---|---|---|---|---|---|---|
| Sequence | Source | (kDa) | #a.a. | pI | Soluble | Heat Stable |
| A | PopA70-146 | 10.69 | 104 | 6.48 | Yes | Yes |
| (N_{N}) | HrpNEa40-80 | 6.754 | 68 | 6.78 | N/A | N/A |
| (N_{N})₂ | Dimer of | 10.84 | 111 | 6.13 | N/A | N/A |
| | HrpNEa40-80 | | | | | |
| (N_{N})₃ | Triplemer of | 14.93 | 154 | 5.63 | N/A | N/A |
| | HrpNEa40-80 | | | | | |
| (N_{N})₄ | Tetramer of | 19.01 | 197 | 4.95 | N/A | N/A |
| | HrpNEa40-80 | | | | | |
| (N_{C}) | HrpNEa140- | 7.224 | 68 | 5.01 | Yes | Yes |
| | 180 | | | | | |
| (N_{C})₂ | Dimer of | 11.78 | 111 | 3.98 | Yes | Yes |
| | HrpNEa140- | | | | | |
| | 180 | | | | | |
| (N_{C})₃ | Triplemer of | 16.34 | 154 | 3.72 | Yes | Yes |
| | HrpNEa140- | | | | | |
| | 180 | | | | | |
| (N_{C})₄ | Tetramer of | 20.89 | 197 | 3.58 | Yes | Yes |
| | HrpNEa140- | | | | | |
| | 180 | | | | | |
| (N_{C})₁₀ | Cancatomer | 48.23 | 455 | 3.28 | N/A | N/A |
| | (10 repeating | | | | | |
| | units of | | | | | |
| | HrpNEa140- | | | | | |
| | 180 | | | | | |
| (N_{C})₁₆ | Cancatomer | 75.57 | 713 | 3.18 | N/A | N/A |
| | (16 repeating | | | | | |
| | units of | | | | | |
| | HrpNEa140- | | | | | |
| | 180 | | | | | |
| W | HrpWEa10-59 | 7.986 | 77 | 6.48 | N/A | N/A |
| Z_{N} | HrpZ90-150 | 8.087 | 78 | 5.38 | Yes | Yes |
| Z₂₆₆₋₃₀₈ | HrpZ266-308 | 7.029 | 70 | 6.40 | Yes | Yes |
| his-tag | | 2.045 | 19 | 11.04 | | |
| leader seq. | | | | | | |

Example 13—Superharpins with Stacked HR Domains and their Biological Activities

There are numerous polypeptides could be generated with different combinations of HR domains or by stacking HR domains and repeating units in order. Selective combination or stacking of HR domains isolated from harpin proteins or other elicitors can be designed to achieve a targeted disease resistance spectrum. See Table 8 for superharpins prepared by stacking of HR building blocks listed on Table 7. All three listed superharpins (i.e. SH-1, SH-2, SH-3) were constructed into a pET28(a) vector and expressed in E. coli. Recombinant proteins were partially purified and quantified by SDS-PAGE with purified Harpin N protein as a quantitative standard.

TABLE 8
Properties of Superharpins

| | | | | | | Heat |
|---|---|---|---|---|---|---|
| Protein | Domain Sequence | MW (kDa) | # a.a. | pI | Soluble | Stable |
| SH-1 | *W(N_{N})₄A(N_{C})₄Z₂₆₆₋₃₀₈ | 54.955 | 545 | 3.69 | Yes | Yes |
| SH-2 | *W(N_{N})₄Z(N_{C})₄Z₂₆₆₋₃₀₈ | 52.341 | 519 | 3.54 | Yes | Yes |
| SH-3 | *W(N_{N})₄A(N_{C})₄Z₂₆₆₋₃₀₈A | 60.375 | 598 | 3.67 | Yes | Yes |
| HrpNEa | HrpN from E.amylovora | 39.697 | 403 | 4.42 | Yes | Yes |

Bioassays for hypersensitive response on tobacco leaves (HR), percentage of TMV reduction on tobacco leaves, and plant growth enhancement with tomato showed that superharpins had higher (up to 2 to 10 fold greater) HR potency compared with HrpN from E. amylovora. This also demonstrated that superharpins have better performance on % TMV reduction and plant growth enhancement assay. See Table 9.

TABLE 9
Biological Activities of Superharpins

| | | Elicit HR | % TMV reduction on tobacco | | % Plant Growth Enhancement | |
|---|---|---|---|---|---|---|
| Protein | Domain Sequence | (˜μg/ml) | 10 μg/ml | 1 μg/ml | 10 μg/ml | 1 μg/ml |
| SH-1 | W(N_{N})₄A(N_{C})₄Z₂₆₆₋₃₀₈ | 0.66 | 83 | 79 | 7.49 | 9.83 |
| SH-2 | W(N_{N})₄Z(N_{C})₄Z₂₆₆₋₃₀₈ | 0.13 | 84 | 60 | 11.05 | 7.30 |
| SH-3 | W(N_{N})₄A(N_{C})₄Z₂₆₆₋₃₀₈A | 0.15 | 77 | 55 | 11.07 | 10.00 |
| HrpNEa | HrpN from E.amylovora | 1—3 | 55 | 10 | 11.68 | N/A |

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. An isolated hypersensitive response elicitor protein comprising an isolated pair or more of spaced apart domains, each comprising an acidic portion linked to an alpha-helix and capable of eliciting a hypersensitive response in plants.

2. A protein according to claim 1, wherein the protein is recombinant.

3. An isolated nucleic acid molecule encoding a protein according to claim 1.

4. A nucleic acid molecule according to claim 3, wherein each domain is from a different source organism.

5. A nucleic acid molecule according to claim 3, wherein there are 3 or more spaced apart domains.

6. An expression vector containing a nucleic acid molecule according to claim 3 which is heterologous to the expression vector.

7. An expression vector according to claim 6, wherein the nucleic acid molecule is positioned in the expression vector in sense orientation and correct reading frame.

8. A host cell transformed with the nucleic acid molecule according to claim 3.

9. A host cell transformed according to claim 8, wherein the host cell is selected from the group consisting of a plant cell, a eukaryotic cell, and a procaryotic cell.

10. A host cell according to claim 8, wherein the nucleic acid molecule is transformed with an expression system.

11. A transgenic plant transformed with the nucleic acid molecule of claim 3.

12. A transgenic plant according to claim 11, wherein the plant is selected from the group consisting of alfalfa, rice, wheat, barley, rye, cotton, sunflower, peanut, corn, potato, sweet potato, bean pea, chicory, lettuce, endive, cabbage, brussel sprout, beet, parsnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

13. A transgenic plant according to claim 11, wherein the plant is selected from the group consisting of Arabidopsis thaliana, Saintpaulia, petunia, pelargonium, poinsettia, chrysanthemum, carnation, and zinnia.

14. A transgenic plant according to claim 11, wherein the plant is a monocot.

15. A transgenic plant according to claim 11, wherein the plant is a dicot.

16. A transgenic plant according to claim 11, wherein each domain is from a different source organism.

17. A transgenic plant according to claim 11, wherein there are 3 or more spaced apart domains.

18. A transgenic plant seed transformed with the nucleic acid molecule of claim 3.

19. A transgenic plant seed according to claim 18, wherein the plant is selected from the group consisting of alfalfa, rice, wheat, barley, rye, cotton, sunflower, peanut, corn, potato, sweet potato, bean pea, chicory, lettuce, endive, cabbage, brussel sprout, beet, parsnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

20. A transgenic plant seed according to claim 18, wherein the plant is selected from the group consisting of Arabidopsis thaliana, Saintpaulia, petunia, pelargonium, poinsettia, chrysanthemum, carnation, and zinnia.

21. A transgenic plant seed according to claim 18, wherein the plant is a monocot.

22. A transgenic plant seed according to claim 18, wherein the plant is a dicot.

23. A method of imparting disease resistance to plants comprising:
applying a protein according to claim 1 to a plant or a plant seed under conditions effective to impart disease resistance to the plant or to a plant grown from the plant seed.

24. A method according to claim 23, wherein the protein is applied to a plant.

25. A method according to claim 23, wherein the protein is applied to a plant seed and further comprising:
planting the plant seed under conditions effective to impart disease resistance to a plant grown from the plant seeds.

26. A method of enhancing plant growth comprising:
applying a protein according to claim 1 to a plant or a plant seed under conditions effective to enhance growth of the plants or of a plant grown from the plant seed.

27. A method according to claim 26, wherein the protein is applied to a plant.

28. A method according to claim 26, wherein the protein is applied to a plant seed and further comprising:
planting the plant seeds under conditions effective to enhance growth of a plant grown from the plant seed.

29. A method of controlling insects comprising:
applying a protein according to claim 1 to a plant or a plant seed under conditions effective to control insects.

30. A method according to claim 29, wherein the protein is applied to a plant.

31. A method according to claim 29, wherein the protein is applied to a plant seed and further comprising:
planting the plant seed under conditions effective to grow a plant from the plant seed and to control insects.

32. A method of imparting stress resistance to plants comprising:
applying a protein according to claim 1 to a plant or a plant seed under conditions effective to impart stress resistance to the plant or to a plant grown from the plant seed.

33. A method according to claim 32, wherein the protein is applied to a plant.

34. A method according to claim 32, wherein the protein is applied to a plant seed and further comprising:
planting the plant seed under conditions effective to impart stress resistance to a plant grown from the plant seed.

35. A method of imparting disease resistance to plants comprising:
providing a transgenic plant or transgenic plant seed containing the nucleic acid according to claim 3 and
planting the transgenic plant or transgenic plant seed under conditions effective to impart disease resistance to the plant or to a plant grown from the plant seed.

36. A method according to claim 35, wherein a transgenic plant is provided.

37. A method according to claim 35, wherein a transgenic plant seed is provided.

38. A method of enhancing growth of plants comprising:
providing a transgenic plant or transgenic plant seed containing the nucleic acid according to claim 3 and
planting the transgenic plant or transgenic plant seed under conditions effective to enhance growth of the plant or of a plant grown from the plant seed.

39. A method according to claim 38, wherein a transgenic plant Is provided.

40. A method according to claim 38, wherein a transgenic plant seed is provided.

41. A method of controlling insects comprising:
providing a transgenic plant or transgenic plant seed containing the nucleic acid according to claim 3 and
planting the transgenic plant or transgenic plant seed under conditions effective to control insects on the plant or on a plant grown from the plant seed.

42. A method according to claim 41, wherein a transgenic plant is provided.

43. A method according to claim 41, wherein a transgenic plant seed is provided.

44. A method of imparting stress resistance to plants comprising:
providing a transgenic plant or transgenic plant seed containing the nucleic acid according to claim 3 and
planting the transgenic plant or transgenic plant seed under conditions effective to impart stress resistance to the plant or to a plant grown from the plant seed.

45. A method according to claim 44, wherein a transgenic plant is provided.

46. A method according to claim 44, wherein a transgenic plant seed is provided.

47. An isolated hypersensitive response elicitor protein comprising, in isolation, a domain comprising an acid portion linked to an alpha-helix and capable of eliciting a hypersensitive response in plants.

48. A protein according to claim 47, wherein the protein is recombinant.

49. An isolated nucleic acid molecule encoding a protein according to claim 47.

50. An isolated nucleic acid molecule according to claim 49, wherein there are at least 2 domains, each from a different source organism.

51. An isolated nucleic acid molecule according to claim 49, wherein there are 3 or more coupled domains.

52. An expression vector containing a nucleic acid molecule according to claim 49 which is heterologous to the expression vector.

53. An expression vector according to claim 52, wherein the nucleic acid molecule is positioned in the expression vector in sense orientation and correct reading frame.

54. A host cell transformed with the nucleic acid molecule according to claim 49.

55. A host cell transformed according to claim 54, wherein the host cell is selected from the group consisting of a plant cell, a eukaryotic cell, and a prokaryotic cell.

56. A host cell according to claim 54, wherein the nucleic acid molecule is transformed with an expression system.

57. A transgenic plant transformed with the nucleic acid molecule of claim 49.

58. A transgenic plant according to claim 57, wherein the plant is selected from the group consisting of alfalfa, rice, wheat, barley, rye, cotton, sunflower, peanut, corn, potato, sweet potato, bean pea, chicory, lettuce, endive, cabbage, brussel sprout, beet, parsnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

59. A transgenic plant according to claim 57, wherein the plant is selected from the group consisting of Arabidopsis thaliana, Saintpaulia, petunia, pelargonium, poinsettia, chrysanthemum, carnation, and zinnia.

60. A transgenic plant according to claim 57, wherein the plant is a monocot.

61. A transgenic plant according to claim 57, wherein the plant is a dicot.

62. A transgenic plant according to claim 57, wherein there are at least 2 coupled domains, each from a different source organism.

63. A transgenic plant according to claim 57, wherein there are 3 or more coupled domains.

64. A transgenic plant seed transformed with the nucleic acid molecule of claim 49.

65. A transgenic plant seed according to claim 64, wherein the plant is selected from the group consisting of alfalfa, rice, wheat, barley, rye, cotton, sunflower, peanut, corn, potato, sweet potato, bean pea, chicory, lettuce, endive, cabbage, brussel sprout, beet, parsnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

66. A transgenic plant seed according to claim 64, wherein the plant is selected from the group consisting of Arabidopsis thaliana, Saintpaulia, petunia, pelargonium, poinsettia, chrysanthemum, carnation, and zinnia.

67. A transgenic plant seed according to claim 64, wherein the plant is a monocot.

68. A transgenic plant seed according to claim 64, wherein the plant is a dicot.

69. A method of imparting disease resistance to plants comprising:
applying a protein according to claim 47 to a plant or a plant seed under conditions effective to impart disease resistance to the plant or to a plant grown from the plant seed.

70. A method according to claim 69, wherein the protein is applied to a plant.

71. A method according to claim 69, wherein the protein is applied to a plant seed and further comprising:
planting the plant seed under conditions effective to impart disease resistance to a plant grown from the plant seed.

72. A method of enhancing plant growth comprising:
applying a protein according to claim 47 to a plant or a plant seed under conditions effective to enhance growth of the plant or of a plant grown from the plant seed.

73. A method according to claim 72, wherein the protein is applied to a plant.

74. A method according to claim 72, wherein the protein is applied to a plant seed and further comprising:
planting the plant seed under conditions effective to enhance growth of a plant grown from the plant seed.

75. A method of controlling insects comprising:
applying a protein according to claim 47 to a plant or a plant seed under conditions effective to control insects.

76. A method according to claim 75, wherein the protein is applied to a plant.

77. A method according to claim 75, wherein the protein is applied to a plant seed and further comprising:
planting the plant seed under conditions effective to grow a plant from the plant seed and to control insects.

78. A method of imparting stress resistance to plants comprising:
applying a protein according to claim 47 to a plant or a plant seed under conditions effective to impart stress resistance to the plant or to a plant grown from the plant seed.

79. A method according to claim 78, wherein the protein is applied to a plant.

80. A method according to claim 78, wherein the protein is applied to a plant seed and further comprising:
planting the plant seed under conditions effective to impart stress resistance to a plant grown from the plant seed.

81. A method of imparting disease resistance to plants comprising:
providing a transgenic plant or transgenic plant seed containing the nucleic acid according to claim 49 and planting the transgenic plant or transgenic plant seed under conditions effective to impart disease resistance to the plant or to a plant grown from the plant seed.

82. A method according to claim 81, wherein a transgenic plant is provided.

83. A method according to claim 81, wherein a transgenic plant seed is provided.

84. A method of enhancing growth of plants comprising:
providing a transgenic plant or transgenic plant seed containing the nucleic acid according to claim 49 and
planting the transgenic plant or transgenic plant seed under conditions effective to enhance growth of the plant or of a plant grown from the plant seed.

85. A method according to claim 84, wherein a transgenic plant is provided.

86. A method according to claim 84, wherein a transgenic plant seed is provided.

87. A method of controlling insects comprising:
providing a transgenic plant or transgenic plant seed containing the nucleic acid according to claim 49 and
planting the transgenic plant or transgenic plant seed under conditions effective to control insects on the plant or on a plant grown from the plant seed.

88. A method according to claim 87, wherein a transgenic plant is provided.

89. A method according to claim 87, wherein a transgenic plant seed is provided.

90. A method of imparting stress resistance to plants comprising:
providing a transgenic plant or transgenic plant seed containing the nucleic acid according to claim 49 and
planting the transgenic plant or transgenic plant seed under conditions effective to impart stress resistance to the plant or to a plant grown from the plant seed.

91. A method according to claim 90, wherein a transgenic plant is provided.

92. A method according to claim 90, wherein a transgenic plant seed is provided.


SEQUENCE LISTING (18 sequences)

SEQ ID NO: 1 - protein, 338 aa, Erwinia chrysanthemi
MQITIKAHIGGDLGVSGLGAQGLKGLNSAASSLGSSVDKLSSTIDKLTSALTSMMFGGALAQGLGASSKGLGMSNQLGQSFGNGAQGASNLLSVPKSGGDALSKMFDKALDDLLGHDTVTKLTNQSNQLANSMLNASQMTQGNMNAFGSGVNNALSSILGNGLGQSMSGFSQPSLGAGGLQGLSGAGAFNQLGNAIGMGVGQNAALSALSNVSTHVDGNNRHFVDKEDRGMAKEIGQFMDQYPEIFGKPEYQKDGWSSPKTDDKSWAKALSKPDDDGMTGASMDKFRQAMGMIKSAVAGDTGNTNLNLRGAGGASLGIDAAVVGDKIANMSLGKLANA

SEQ ID NO: 2 - DNA, 2141 bp, Erwinia chrysanthemi
cgattttacccgggtgaacgtgctatgaccgacagcatcacggtattcgacaccgttacggcgtttatggccgcgatgaaccggcatcaggcggcgcgctggtcgccgcaatccggcgtcgatctggtatttcagtttggggacaccgggcgtgaactcatgatgcagattcagccggggcagcaatatcccggcatgttgcgcacgctgctcgctcgtcgttatcagcaggcggcagagtgcgatggctgccatctgtgcctgaacggcagcgatgtattgatcctctggtggccgctgccgtcggatcccggcagttatccgcaggtgatcgaacgtttgtttgaactggcgggaatgacgttgccgtcgctatccatagcaccgacggcgcgtccgcagacagggaacggacgcgcccgatcattaagataaaggcggctttttttattgcaaaacggtaacggtgaggaaccgtttcaccgtcggcgtcactcagtaacaagtatccatcatgatgcctacatcgggatcggcgtgggcatccgttgcagatacttttgcgaacacctgacatgaatgaggaaacgaaattatgcaaattacgatcaaagcgcacatcggcggtgatttgggcgtctccggtctggggctgggtgctcagggactgaaaggactgaattccgcggcttcatcgctgggttccagcgtggataaactgagcagcaccatcgataagttgacctccgcgctgacttcgatgatgtttggcggcgcgctggcgcaggggctgggcgccagctcgaaggggctggggatgagcaatcaactgggccagtctttcggcaatggcgcgcagggtgcgagcaacctgctatccgtaccgaaatccggcggcgatgcgttgtcaaaaatgtttgataaagcgctggacgatctgctgggtcatgacaccgtgaccaagctgactaaccagagcaaccaactggctaattcaatgctgaacgccagccagatgacccagggtaatatgaatgcgttcggcagcggtgtgaacaacgcactgtcgtccattctcggcaacggtctcggccagtcgatgagtggcttctctcagccttctctgggggcaggcggcttgcagggcctgagcggcgcgggtgcattcaaccagttgggtaatgccatcggcatgggcgtggggcagaatgctgcgctgagtgcgttgagtaacgtcagcacccacgtagacggtaacaaccgccactttgtagataaagaagatcgcggcatggcgaaagagatcggccagtttatggatcagtatccggaaatattcggtaaaccggaataccagaaagatggctggagttcgccgaagacggacgacaaatcctgggctaaagcgctgagtaaaccggatgatgacggtatgaccggcgccagcatggacaaattccgtcaggcgatgggtatgatcaaaagcgcggtggcgggtgataccggcaataccaacctgaacctgcgtggcgcgggcggtgcatcgctgggtatcgatgcggctgtcgtcggcgataaaatagccaacatgtcgctgggtaagctggccaacgcctgataatctgtgctggcctgataaagcggaaacgaaaaaagagacggggaagcctgtctcttttcttattatgcggtttatgcggttacctggaccggttaatcatcgtcatcgatctggtacaaacgcacattttcccgttcattcgcgtcgttacgcgccacaatcgcgatggcatcttcctcgtcgctcagattgcgcggctgatggggaacgccgggtggaatatagagaaactcgccggccagatggagacacgtctgcgataaatctgtgccgtaacgtgtttctatccgcccctttagcagatagattgcggtttcgtaatcaacatggtaatgcggttccgcctgtgcgccggccgggatcaccacaatattcatagaaagctgtcttgcacctaccgtatcgcgggagataccgacaaaatagggcagtttttgcgtggtatccgtggggtgttccggcctgacaatcttgagttggttcgtcatcatctttctccatctgggcgacctgatcggtt

SEQ ID NO: 3 - protein, 403 aa, Erwinia amylovora
MSLNTSGLGASTMQISIGGAGGNNGLLGTSRQNAGLGGNSALGLGGGNQNDTVNQLAGLLTGMMMMMSMMGGGGLMGGGLGGGLGNGLGGSGGLGEGLSNALNDMLGGSLNTLGSKGGNNTTSTTNSPLDQALGINSTSQNDDSTSGTDSTSDSSDPMQQLLKMFSEIMQSLFGDGQDGTQGSSSGGKQPTEGEQNAYKKGVTDALSGLMGNGLSQLLGNGGLGGGQGGNAGTGLDGSSLGGKGLQNLSGPVDYQQLGNAVGTGIGMKAGIQALNDIGTHRHSSTRSFVNKGDRAMAKEIGQFMDQYPEVFGKPQYQKGPGQEVKTDDKSWAKALSKPDDDGMTPASMEQFNKAKGMIKRPMAGDTGNGNLQARGAGGSSLGIDAMMAGDAINNMALGKLGAA

SEQ ID NO: 4 - DNA, 1288 bp, Erwinia amylovora
aagcttcggcatggcacgtttgaccgttgggtcggcagggtacgtttgaattattcataagaggaatacgttatgagtctgaatacaagtgggctgggagcgtcaacgatgcaaatttctatcggcggtgcgggcggaaataacgggttgctgggtaccagtcgccagaatgctgggttgggtggcaattctgcactggggctgggcggcggtaatcaaaatgataccgtcaatcagctggctggcttactcaccggcatgatgatgatgatgagcatgatgggcggtggtgggctgatgggcggtggcttaggcggtggcttaggtaatggcttgggtggctcaggtggcctgggcgaaggactgtcgaacgcgctgaacgatatgttaggcggttcgctgaacacgctgggctcgaaaggcggcaacaataccacttcaacaacaaattccccgctggaccaggcgctgggtattaactcaacgtcccaaaacgacgattccacctccggcacagattccacctcagactccagcgacccgatgcagcagctgctgaagatgttcagcgagataatgcaaagcctgtttggtgatgggcaagatggcacccagggcagttcctctgggggcaagcagccgaccgaaggcgagcagaacgcctataaaaaaggagtcactgatgcgctgtcgggcctgatgggtaatggtctgagccagctccttggcaacgggggactgggaggtggtcagggcggtaatgctggcacgggtcttgacggttcgtcgctgggcggcaaagggctgcaaaacctgagcgggccggtggactaccagcagttaggtaacgccgtgggtaccggtatcggtatgaaagcgggcattcaggcgctgaatgatatcggtacgcacaggcacagttcaacccgttctttcgtcaataaaggcgatcgggcgatggcgaaggaaatcggtcagttcatggaccagtatcctgaggtgtttggcaagccgcagtaccagaaaggcccgggtcaggaggtgaaaaccgatgacaaatcatgggcaaaagcactgagcaagccagatgacgacggaatgacaccagccagtatggagcagttcaacaaagccaagggcatgatcaaaaggcccatggcgggtgataccggcaacggcaacctgcaggcacgcggtgccggtggttcttcgctgggtattgatgccatgatggccggtgatgccattaacaatatggcacttggcaagctgggcgcggcttaagctt

SEQ ID NO: 5 - DNA, 1344 bp, Erwinia amylovora
atgtcaattcttacgcttaacaacaatacctcgtcctcgccgggtctgttccagtccgggggggacaacgggcttggtggtcataatgcaaattctgcgttggggcaacaacccatcgatcggcaaaccattgagcaaatggctcaattattggcggaactgttaaagtcactgctatcgccacaatcaggtaatgcggcaaccggagccggtggcaatgaccagactacaggagttggtaacgctggcggcctgaacggacgaaaaggcacagcaggaaccactccgcagtctgacagtcagaacatgctgagtgagatgggcaacaacgggctggatcaggccatcacgcccgatggccagggcggcgggcagatcggcgataatcctttactgaaagccatgctgaagcttattgcacgcatgatggacggccaaagcgatcagtttggccaacctggtacgggcaacaacagtgcctcttccggtacttcttcatctggcggttccccttttaacgatctatcaggggggaaggccccttccggcaactccccttccggcaactactctcccgtcagtaccttctcacccccatccacgccaacgtcccctacctcaccgcttgatttcccttcttctcccaccaaagcagccgggggcagcacgccggtaaccgatcatcctgaccctgttggtagcgcgggcatcggggccggaaattcggtggccttcaccagcgccggcgctaatcagacggtgctgcatgacaccattaccgtgaaagcgggtcaggtgtttgatggcaaaggacaaaccttcaccgccggttcagaattaggcgatggcggccagtctgaaaaccagaaaccgctgtttatactggaagacggtgccagcctgaaaaacgtcaccatgggcgacgacggggcggatggtattcatctttacggtgatgccaaaatagacaatctgcacgtcaccaacgtgggtgaggacgcgattaccgttaagccaaacagcgcgggcaaaaaatcccacgttgaaatcactaacagttccttcgagcacgcctctgacaagatcctgcagctgaatgccgatactaacctgagcgttgacaacgtgaaggccaaagactttggtacttttgtacgcactaacggcggtcaacagggtaactgggatctgaatctgagccatatcagcgcagaagacggtaagttctcgttcgttaaaagcgatagcgaggggctaaacgtcaataccagtgatatctcactgggtgatgttgaaaaccactacaaagtgccgatgtccgccaacctgaaggtggctgaatga

SEQ ID NO: 6 - protein, 447 aa, Erwinia amylovora
MSILTLNNNTSSSPGLFQSGGDNGLGGHNANSALGQQPIDRQTIEQMAQLLAELLKSLLSPQSGNAATGAGGNDQTTGVGNAGGLNGRKGTAGTTPQSDSQNMLSEMGNNGLDQAITPDGQGGGQIGDNPLLKAMLKLIARMMDGQSDQFGQPGTGNNSASSGTSSSGGSPFNDLSGGKAPSGNSPSGNYSPVSTFSPPSTPTSPTSPLDFPSSPTKAAGGSTPVTDHPDPVGSAGIGAGNSVAFTSAGANQTVLHDTITVKAGQVFDGKGQTFTAGSELGDGGQSENQKPLFILEDGASLKNVTMGDDGADGIHLYGDAKIDNLHVTNVGEDAITVKPNSAGKKSHVEITNSSFEHASDKILQLNADTNLSVDNVKAKDFGTFVRTNGGQQGNWDLNLSHISAEDGKFSFVKSDSEGLNVNTSDISLGDVENHYKVPMSANLKVAE

SEQ ID NO: 7 - DNA, 5517 bp, Erwinia amylovora
atggaattaaaatcactgggaactgaacacaaggcggcagtacacacagcggcgcacaaccctgtggggcatggtgttgccttacagcagggcagcagcagcagcagcccgcaaaatgccgctgcatcattggcggcagaaggcaaaaatcgtgggaaaatgccgagaattcaccagccatctactgcggctgatggtatcagcgctgctcaccagcaaaagaaatccttcagtctcaggggctgtttggggacgaaaaaattttccagatcggcaccgcagggccagccaggtaccacccacagcaaaggggcaacattgcgcgatctgctggcgcgggacgacggcgaaacgcagcatgaggcggccgcgccagatgcggcgcgtttgacccgttcgggcggcgtcaaacgccgcaatatggacgacatggccgggcggccaatggtgaaaggtggcagcggcgaagataaggtaccaacgcagcaaaaacggcatcagctgaacaattttggccagatgcgccaaacgatgttgagcaaaatggctcacccggcttcagccaacgccggcgatcgcctgcagcattcaccgccgcacatcccgggtagccaccacgaaatcaaggaagaaccggttggctccaccagcaaggcaacaacggcccacgcagacagagtggaaatcgctcaggaagatgacgacagcgaattccagcaactgcatcaacagcggctggcgcgcgaacgggaaaatccaccgcagccgcccaaactcggcgttgccacaccgattagcgccaggtttcagcccaaactgactgcggttgcggaaagcgtccttgaggggacagataccacgcagtcaccccttaagccgcaatcaatgctgaaaggaagtggagccggggtaacgccgctggcggtaacgctggataaaggcaagttgcagctggcaccggataatccacccgcgctcaatacgttgttgaagcagacattgggtaaagacacccagcactatctggcgcaccatgccagcagcgacggtagccagcatctgctgctggacaacaaaggccacctgtttgatatcaaaagcaccgccaccagctatagcgtgctgcacaacagccaccccggtgagataaagggcaagctggcgcaggcgggtactggctccgtcagcgtagacggtaaaagcggcaagatctcgctggggagcggtacgcaaagtcacaacaaaacaatgctaagccaaccgggggaagcgcaccgttccttattaaccggcatttggcagcatcctgctggcgcagcgcggccgcagggcgagtcaatccgcctgcatgacgacaaaattcatatcctgcatccggagctgggcgtatggcaatctgcggataaagatacccacagccagctgtctcgccaggcagacggtaagctctatgcgctgaaagacaaccgtaccctgcaaaacctctccgataataaatcctcagaaaagctggtcgataaaatcaaatcgtattccgttgatcagcgggggcaggtggcgatcctgacggatactcccggccgccataagatgagtattatgccctcgctggatgcttccccggagagccatatttccctcagcctgcattttgccgatgcccaccaggggttattgcacgggaagtcggagcttgaggcacaatctgtcgcgatcagccatgggcgactggttgtggccgatagcgaaggcaagctgtttagcgccgccattccgaagcaaggggatggaaacgaactgaaaatgaaagccatgcctcagcatgcgctcgatgaacattttggtcatgaccaccagatttctggatttttccatgacgaccacggccagcttaatgcgctggtgaaaaataacttcaggcagcagcatgcctgcccgttgggtaacgatcatcagtttcaccccggctggaacctgactgatgcgctggttatcgacaatcagctggggctgcatcataccaatcctgaaccgcatgagattcttgatatggggcatttaggcagcctggcgttacaggagggcaagcttcactattttgaccagctgaccaaagggtggactggcgcggagtcagattgtaagcagctgaaaaaaggcctggatggagcagcttatctactgaaagacggtgaagtgaaacgcctgaatattaatcagagcacctcctctatcaagcacggaacggaaaacgttttttcgctgccgcatgtgcgcaataaaccggagccgggagatgccctgcaagggctgaataaagacgataaggcccaggccatggcggtgattggggtaaataaatacctggcgctgacggaaaaaggggacattcgctccttccagataaaacccggcacccagcagttggagcggccggcacaaactctcagccgcgaaggtatcagcggcgaactgaaagacattcatgtcgaccacaagcagaacctgtatgccttgacccacgagggagaggtgtttcatcagccgcgtgaagcctggcagaatggtgccgaaagcagcagctggcacaaactggcgttgccacagagtgaaagtaagctaaaaagtctggacatgagccatgagcacaaaccgattgccacctttgaagacggtagccagcatcagctgaaggctggcggctggcacgcctatgcggcacctgaacgcgggccgctggcggtgggtaccagcggttcacaaaccgtctttaaccgactaatgcagggggtgaaaggcaaggtgatcccaggcagcgggttgacggttaagctctcggctcagacggggggaatgaccggcgccgaagggcgcaaggtcagcagtaaattttccgaaaggatccgcgcctatgcgttcaacccaacaatgtccacgccgcgaccgattaaaaatgctgcttatgccacacagcacggctggcaggggcgtgaggggttgaagccgttgtacgagatgcagggagcgctgattaaacaactggatgcgcataacgttcgtcataacgcgccacagccagatttgcagagcaaactggaaactctggatttaggcgaacatggcgcagaattgcttaacgacatgaagcgcttccgcgacgaactggagcagagtgcaacccgttcggtgaccgttttaggtcaacatcagggagtgctaaaaagcaacggtgaaatcaatagcgaatttaagccatcgcccggcaaggcgttggtccagagctttaacgtcaatcgctctggtcaggatctaagcaagtcactgcaacaggcagtacatgccacgccgccatccgcagagagtaaactgcaatccatgctggggcactttgtcagtgccggggtggatatgagtcatcagaagggcgagatcccgctgggccgccagcgcgatccgaatgataaaaccgcactgaccaaatcgcgtttaattttagataccgtgaccatcggtgaactgcatgaactggccgataaggcgaaactggtatctgaccataaacccgatgccgatcagataaaacagctgcgccagcagttcgatacgctgcgtgaaaagcggtatgagagcaatccggtgaagcattacaccgatatgggcttcacccataataaggcgctggaagcaaactatgatgcggtcaaagcctttatcaatgcctttaagaaagagcaccacggcgtcaatctgaccacgcgtaccgtactggaatcacagggcagtgcggagctggcgaagaagctcaagaatacgctgttgtccctggacagtggtgaaagtatgagcttcagccggtcatatggcgggggcgtcagcactgtctttgtgcctacccttagcaagaaggtgccagttccggtgatccccggagccggcatcacgctggatcgcgcctataacctgagcttcagtcgtaccagcggcggattgaacgtcagttttggccgcgacggcggggtgagtggtaacatcatggtcgctaccggccatgatgtgatgccctatatgaccggtaagaaaaccagtgcaggtaacgccagtgactggttgagcgcaaaacataaaatcagcccggacttgcgtatcggcgctgctgtgagtggcaccctgcaaggaacgctacaaaacagcctgaagtttaagctgacagaggatgagctgcctggctttatccatggcttgacgcatggcacgttgaccccggcagaactgttgcaaaaggggatcgaacatcagatgaagcagggcagcaaactgacgtttagcgtcgatacctcggcaaatctggatctgcgtgccggtatcaatctgaacgaagacggcagtaaaccaaatggtgtcactgcccgtgtttctgccgggctaagtgcatcggcaaacctggccgccggctcgcgtgaacgcagcaccacctctggccagtttggcagcacgacttcggccagcaataaccgcccaaccttcctcaacggggtcggcgcgggtgctaacctgacggctgctttaggggttgcccattcatctacgcatgaagggaaaccggtcgggatcttcccggcatttacctcgaccaatgtttcggcagcgctggcgctggataaccgtacctcacagagtatcagcctggaattgaagcgcgcggagccggtgaccagcaacgatatcagcgagttgacctccacgctgggaaaacactttaaggatagcgccacaacgaagatgcttgccgctctcaaagagttagatgacgctaagcccgctgaacaactgcatattttacagcagcatttcagtgcaaaagatgtcgtcggtgatgaacgctacgaggcggtgcgcaacctgaaaaaactggtgatacgtcaacaggctgcggacagccacagcatggaattaggatctgccagtcacagcacgacctacaataatctgtcgagaataaataatgacggcattgtcgagctgctacacaaacatttcgatgcggcattaccagcaagcagtgccaaacgtcttggtgaaatgatgaataacgatccggcactgaaagatattattaagcagctgcaaagtacgccgttcagcagcgccagcgtgtcgatggagctgaaagatggtctgcgtgagcagacggaaaaagcaatactggacggtaaggtcggtcgtgaagaagtgggagtacttttccaggatcgtaacaacttgcgtgttaaatcggtcagcgtcagtcagtccgtcagcaaaagcgaaggcttcaataccccagcgctgttactggggacgagcaacagcgctgctatgagcatggagcgcaacatcggaaccattaattttaaatacggccaggatcagaacaccccacggcgatttaccctggagggtggaatagctcaggctaatccgcaggtcgcatctgcgcttactgatttgaagaaggaagggctggaaatgaagagctaa

SEQ ID NO: 8 - protein, 1838 aa, Erwinia amylovora
MELKSLGTEHKAAVHTAAHNPVGHGVALQQGSSSSSPQNAAASLAAEGKNRGKMPRIHQPSTAADGISAAHQQKKSFSLRGCLGTKKFSRSAPQGQPGTTHSKGATLRDLLARDDGETQHEAAAPDAARLTRSGGVKRRNMDDMAGRPMVKGGSGEDKVPTQQKRHQLNNFGQMRQTMLSKMAHPASANAGDRLQHSPPHIPGSHHEIKEEPVGSTSKATTAHADRVEIAQEDDDSEFQQLHQQRLARERENPPQPPKLGVATPISARFQPKLTAVAESVLEGTDTTQSPLKPQSMLKGSGAGVTPLAVTLDKGKLQLAPDNPPALNTLLKQTLGKDTQHYLAHHASSDGSQHLLLDNKGHLFDIKSTATSYSVLHNSHPGEIKGKLAQAGTGSVSVDGKSGKISLGSGTQSHNKTMLSQPGEAHRSLLTGIWQHPAGAARPQGESIRLHDDKIHILHPELGVWQSADKDTHSQLSRQADGKLYALKDNRTLQNLSDNKSSEKLVDKIKSYSVDQRGQVAILTDTPGRHKMSIMPSLDASPESHISLSLHFADAHQGLLHGKSELEAQSVAISHGRLVVADSEGKLFSAAIPKQGDGNELKMKAMPQHALDEHFGHDHQISGFFHDDHGQLNALVKNNFRQQHACPLGNDHQFHPGWNLTDALVIDNQLGLHHTNPEPHEILDMGHLGSLALQEGKLHYFDQLTKGWTGAESDCKQLKKGLDGAAYLLKDGEVKRLNINQSTSSIKHGTENVFSLPHVRNKPEPGDALQGLNKDDKAQAMAVIGVNKYLALTEKGDIRSFQIKPGTQQLERPAQTLSREGISGELKDIHVDHKQNLYALTHEGEVFHQPREAWQNGAESSSWHKLALPQSESKLKSLDMSHEHKPIATFEDGSQHQLKAGGWHAYAAPERGPLAVGTSGSQTVFNRLMQGVKGKVIPGSGLTVKLSAQTGGMTGAEGRKVSSKFSERIRAYAFNPTMSTPRPIKNAAYATQHGWQGREGLKPLYEMQGALIKQLDAHNVRHNAPQPDLQSKLETLDLGEHGAELLNDMKRFRDELEQSATRSVTVLGQHQGVLKSNGEINSEFKPSPGKALVQSFNVNRSGQDLSKSLQQAVHATPPSAESKLQSMLGHFVSAGVDMSHQKGEIPLGRQRDPNDKTALTKSRLILDTVTIGELHELADKAKLVSDHKPDADQIKQLRQQFDTLREKRYESNPVKHYTDMGFTHNKALEANYDAVKAFINAFKKEHHGVNLTTRTVLESQGSAELAKKLKNTLLSLDSGESMSFSRSYGGGVSTVFVPTLSKKVPVPVIPGAGITLDRAYNLSFSRTSGGLNVSFGRDGGVSGNIMVATGHDVMPYMTGKKTSAGNASDWLSAKHKISPDLRIGAAVSGTLQGTLQNSLKFKLTEDELPGFIHGLTHGTLTPAELLQKGIEHQMKQGSKLTFSVDTSANLDLRAGINLNEDGSKPNGVTARVSAGLSASANLAAGSRERSTTSGQFGSTTSASNNRPTFLNGVGAGANLTAALGVAHSSTHEGKPVGIFPAFTSTNVSAALALDNRTSQSISLELKRAEPVTSNDISELTSTLGKHFKDSATTKMLAALKELDDAKPAEQLHILQQHFSAKDVVGDERYEAVRNLKKLVIRQQAADSHSMELGSASHSTTYNNLSRINNDGIVELLHKHFDAALPASSAKRLGEMMNNDPALKDIIKQLQSTPFSSASVSMELKDGLREQTEKAILDGKVGREEVGVLFQDRNNLRVKSVSVSQSVSKSEGFNTPALLLGTSNSAAMSMERNIGTINFKYGQDQNTPRRFTLEGGIAQANPQVASALTDLKKEGLEMKS

SEQ ID NO: 9 - DNA, 420 bp, Erwinia amylovora
atgacatcgtcacagcagcgggttgaaaggtttttacagtatttctccgccgggtgtaaaacgcccatacatctgaaagacggggtgtgcgccctgtataacgaacaagatgaggaggcggcggtgctggaagtaccgcaacacagcgacagcctgttactacactgccgaatcattgaggctgacccacaaacttcaataaccctgtattcgatgctattacagctgaattttgaaatggcggccatgcgcggctgttggctggcgctggatgaactgcacaacgtgcgtttatgttttcagcagtcgctggagcatctggatgaagcaagttttagcgatatcgttagcggcttcatcgaacatgcggcagaagtgcgtgagtatatagcgcaattagacgagagtagcgcggcataa

SEQ ID NO: 10 - protein, 139 aa, Erwinia amylovora
MTSSQQRVERFLQYFSAGCKTPIHLKDGVCALYNEQDEEAAVLEVPQHSDSLLLHCRIIEADPQTSITLYSMLLQLNFEMAAMRGCWLALDELHNVRLCFQQSLEHLDEASFSDIVSGFIEHAAEVREYIAQLDESSAA

SEQ ID NO: 11 - protein, 341 aa, Pseudomonas syringae
MQSLSLNSSSLQTPAMALVLVRPEAETTGSTSSKALQEVVVKLAEELMRNGQLDDSSPLGKLLAKSMAADGKAGGGIEDVIAALDKLIHEKLGDNFGASADSASGTGQQDLMTQVLNGLAKSMLDDLLTKQDGGTSFSEDDMPMLNKIAQFMDDNPAQFPKPDSGSWVNELKEDNFLDGDETAAFRSALDIIGQQLGNQQSDAGSLAGTGGGLGTPSSFSNNSSVMGDPLIDANTGPGDSGNTRGEAGQLIGELIDRGLQSVLAGGGLGTPVNTPQTGTSANGGQSAQDLDQLLGGLLLKGLEATLKDAGQTGTDVQSSAAQIATLLVSTLLQGTRNQAAA

SEQ ID NO: 12 - DNA, 1026 bp, Pseudomonas syringae
atgcagagtctcagtcttaacagcagctcgctgcaaaccccggcaatggcccttgtcctggtacgtcctgaagccgagacgactggcagtacgtcgagcaaggcgcttcaggaagttgtcgtgaagctggccgaggaactgatgcgcaatggtcaactcgacgacagctcgccattgggaaaactgttggccaagtcgatggccgcagatggcaaggcgggcggcggtattgaggatgtcatcgctgcgctggacaagctgatccatgaaaagctcggtgacaacttcggcgcgtctgcggacagcgcctcgggtaccggacagcaggacctgatgactcaggtgctcaatggcctggccaagtcgatgctcgatgatcttctgaccaagcaggatggcgggacaagcttctccgaagacgatatgccgatgctgaacaagatcgcgcagttcatggatgacaatcccgcacagtttcccaagccggactcgggctcctgggtgaacgaactcaaggaagacaacttccttgatggcgacgaaacggctgcgttccgttcggcactcgacatcattggccagcaactgggtaatcagcagagtgacgctggcagtctggcagggacgggtggaggtctgggcactccgagcagtttttccaacaactcgtccgtgatgggtgatccgctgatcgacgccaataccggtcccggtgacagcggcaatacccgtggtgaagcggggcaactgatcggcgagcttatcgaccgtggcctgcaatcggtattggccggtggtggactgggcacacccgtaaacaccccgcagaccggtacgtcggcgaatggcggacagtccgctcaggatcttgatcagttgctgggcggcttgctgctcaagggcctggaggcaacgctcaaggatgccgggcaaacaggcaccgacgtgcagtcgagcgctgcgcaaatcgccaccttgctggtcagtacgctgctgcaaggcacccgcaatcaggctgcagcctga

SEQ ID NO: 13 - DNA, 1729 bp, Pseudomonas syringae
tccacttcgctgattttgaaattggcagattcatagaaacgttcaggtgtggaaatcaggctgagtgcgcagatttcgttgataagggtgtggtactggtcattgttggtcatttcaaggcctctgagtgcggtgcggagcaataccagtcttcctgctggcgtgtgcacactgagtcgcaggcataggcatttcagttccttgcgttggttgggcatataaaaaaaggaacttttaaaaacagtgcaatgagatgccggcaaaacgggaaccggtcgctgcgctttgccactcacttcgagcaagctcaaccccaaacatccacatccctatcgaacggacagcgatacggccacttgctctggtaaaccctggagctggcgtcggtccaattgcccacttagcgaggtaacgcagcatgagcatcggcatcacaccccggccgcaacagaccaccacgccactcgatttttcggcgctaagcggcaagagtcctcaaccaaacacgttcggcgagcagaacactcagcaagcgatcgacccgagtgcactgttgttcggcagcgacacacagaaagacgtcaacttcggcacgcccgacagcaccgtccagaatccgcaggacgccagcaagcccaacgacagccagtccaacatcgctaaattgatcagtgcattgatcatgtcgttgctgcagatgctcaccaactccaataaaaagcaggacaccaatcaggaacagcctgatagccaggctcctttccagaacaacggcgggctcggtacaccgtcggccgatagcgggggcggcggtacaccggatgcgacaggtggcggcggcggtgatacgccaagcgcaacaggcggtggcggcggtgatactccgaccgcaacaggcggtggcggcagcggtggcggcggcacacccactgcaacaggtggcggcagcggtggcacacccactgcaacaggcggtggcgagggtggcgtaacaccgcaaatcactccgcagttggccaaccctaaccgtacctcaggtactggctcggtgtcggacaccgcaggttctaccgagcaagccggcaagatcaatgtggtgaaagacaccatcaaggtcggcgctggcgaagtctttgacggccacggcgcaaccttcactgccgacaaatctatgggtaacggagaccagggcgaaaatcagaagcccatgttcgagctggctgaaggcgctacgttgaagaatgtgaacctgggtgagaacgaggtcgatggcatccacgtgaaagccaaaaacgctcaggaagtcaccattgacaacgtgcatgcccagaacgtcggtgaagacctgattacggtcaaaggcgagggaggcgcagcggtcactaatctgaacatcaagaacagcagtgccaaaggtgcagacgacaaggttgtccagctcaacgccaacactcacttgaaaatcgacaacttcaaggccgacgatttcggcacgatggttcgcaccaacggtggcaagcagtttgatgacatgagcatcgagctgaacggcatcgaagctaaccacggcaagttcgccctggtgaaaagcgacagtgacgatctgaagctggcaacgggcaacatcgccatgaccgacgtcaaacacgcctacgataaaacccaggcatcgacccaacacaccgagctttgaatccagacaagtagcttgaaaaaagggggtggactc

SEQ ID NO: 14 - protein, 424 aa, Pseudomonas syringae
MSIGITPRPQQTTTPLDFSALSGKSPQPNTFGEQNTQQAIDPSALLFGSDTQKDVNFGTPDSTVQNPQDASKPNDSQSNIAKLISALIMSLLQMLTNSNKKQDTNQEQPDSQAPFQNNGGLGTPSADSGGGGTPDATGGGGGDTPSATGGGGGDTPTATGGGGSGGGGTPTATGGGSGGTPTATGGGEGGVTPQITPQLANPNRTSGTGSVSDTAGSTEQAGKINVVKDTIKVGAGEVFDGHGATFTADKSMGNGDQGENQKPMFELAEGATLKNVNLGENEVDGIHVKAKNAQEVTIDNVHAQNVGEDLITVKGEGGAAVTNLNIKNSSAKGADDKVVQLNANTHLKIDNFKADDFGTMVRTNGGKQFDDMSIELNGIEANHGKFALVKSDSDDLKLATGNIAMTDVKHAYDKTQASTQHTEL

SEQ ID NO: 15 - protein, 344 aa, Pseudomonas solanacearum
MSVGNIQSPSNLPGLQNLNLNTNTNSQQSGQSVQDLIKQVEKDILNIIAALVQKAAQSAGGNTGNTGNAPAKDGNANAGANDPSKNDPSKSQAPQSANKTGNVDDANNQDPMQALMQLLEDLVKLLKAALHMQQPGGNDKGNGVGGANGAKGAGGQGGLAEALQEIEQILAQLGGGGAGAGGAGGGVGGAGGADGGSGAGGAGGANGADGGNGVNGNQANGPQNAGDVNGANGADDGSEDQGGLTGVLQKLMKILNALVQMMQQGGLGGGNQAQGGSKGAGNASPASGANPGANQPGSADDQSSGQNNLQSQIMDVVKEVVQILQQMLAAQNGGSQQSTSTQPM

SEQ ID NO: 16 - DNA, 1035 bp, Pseudomonas solanacearum
atgtcagtcggaaacatccagagcccgtcgaacctcccgggtctgcagaacctgaacctcaacaccaacaccaacagccagcaatcgggccagtccgtgcaagacctgatcaagcaggtcgagaaggacatcctcaacatcatcgcagccctcgtgcagaaggccgcacagtcggcgggcggcaacaccggtaacaccggcaacgcgccggcgaaggacggcaatgccaacgcgggcgccaacgacccgagcaagaacgacccgagcaagagccaggctccgcagtcggccaacaagaccggcaacgtcgacgacgccaacaaccaggatccgatgcaagcgctgatgcagctgctggaagacctggtgaagctgctgaaggcggccctgcacatgcagcagcccggcggcaatgacaagggcaacggcgtgggcggtgccaacggcgccaagggtgccggcggccagggcggcctggccgaagcgctgcaggagatcgagcagatcctcgcccagctcggcggcggcggtgctggcgccggcggcgcgggtggcggtgtcggcggtgctggtggcgcggatggcggctccggtgcgggtggcgcaggcggtgcgaacggcgccgacggcggcaatggcgtgaacggcaaccaggcgaacggcccgcagaacgcaggcgatgtcaacggtgccaacggcgcggatgacggcagcgaagaccagggcggcctcaccggcgtgctgcaaaagctgatgaagatcctgaacgcgctggtgcagatgatgcagcaaggcggcctcggcggcggcaaccaggcgcagggcggctcgaagggtgccggcaacgcctcgccggcttccggcgcgaacccgggcgcgaaccagcccggttcggcggatgatcaatcgtccggccagaacaatctgcaatcccagatcatggatgtggtgaaggaggtcgtccagatcctgcagcagatgctggcggcgcagaacggcggcagccagcagtccacctcgacgcagccgatgtaa

SEQ ID NO: 17 - protein, 10 aa, Xanthomonas campestris
MDGIGNHFSN

SEQ ID NO: 18 - protein, 20 aa, Xanthomonas campestris pv. pelargonii
SSQQSPSAGSEQQLDQLLAM